(19) United States
(12) Patent Application Publication
(10) Pub. No.: US 2013/0245233 A1
(43) Pub. Date: Sep. 19, 2013

(54) Multispecific Molecules

(71) Applicants: Ming Lei, Acton, MA (US); Jing Li, Lexington, MA (US); Li Zhou, West Roxbury, MA (US); Zhenping Zhu, New York, NY (US)

(72) Inventors: Ming Lei, Acton, MA (US); Jing Li, Lexington, MA (US); Li Zhou, West Roxbury, MA (US); Zhenping Zhu, New York, NY (US)

(21) Appl. No.: 13/988,802

(22) PCT Filed: Nov. 22, 2011
(86) PCT No.: PCT/EP2011/070668

§ 371 Date: May 22, 2013

Related U.S. Application Data
(60) Provisional application No. 61/416,839, filed on Nov. 24, 2010.

(51) Int. Cl. C07K 16/46

(52) U.S. Cl. CPC C07K 16/468
USPC 530/387.3

(57) ABSTRACT

The present invention is directed to multi-specific/multivalent molecules of novel formats. The molecules with the formats of the present invention have desired yield and thermostability. The present invention also includes methods of producing and using the molecules described herein.

### FIELD OF THE INVENTION

The present invention relates to the field of immunology. Specifically, the invention relates to novel formats of multispecific and/or multivalent molecules and methods of making and using thereof.

### BACKGROUND OF THE INVENTION

Multi-specific molecules that are capable of binding two or more antigens are known in the art and offer significant clinical benefits, for example, for diagnostic enzyme assays, as a vaccine adjuvant, for delivering thrombolytic agents, for treating infectious diseases, for targeting immune complexes to cell surface receptors, or for delivering immunotoxins to tumor cells, etc. (Burrows, F. J. and Thorpe, P. E. (1993) Proc Natl Acad Sci USA 90:8996 9000; Zhu, Z. et al. (1996) Bio/Technology 14:192 196; Holliger, P. et al. (1996) Protein Engin. 9:299 305; Morrison et al., (1997) Nature Biotech. 15:159-163; Huang, X. et al. (1997) Science 275:547 550Alt et al. (1999) FEBS Letters 454: 90-94; Zuo et al., (2000) Protein Engineering 13:361-367; Lu et al., (2004) JBC 279:2856-2865; Lu et al., (2005) JBC 280:19665-19672; Marvin et al., (2005) Acta Pharmacologica Sinica 26:649-658; Marvin et al., (2006) Curr Opin Drug Disc Develop 9:184-193; Shen et al., (2007) J Immun Methods 218:65-74; Wu et al., (2007) Nat Biotechnol. 11:1290-1297; Dimasi et al., (2009) J Mol Biol. 393:672-692; and Michaelson et al., (2009) mAbs 1:128-141).

Multi-specific molecules derived from antibodies can be generated using cell fusion, chemical conjugation, or recombinant DNA techniques. A wide variety of multi-specific molecule formats have been developed in the art. One kind of formats does not contain CH2 and CH3 domains, where such formats comprise antigen-binding moieties of one antibody connected in a desired pattern with binding domains of one or more other antibodies/binding molecules. Although this kind of formats has dual or multiple binding specificities with the desired antigens, they no longer retain an antibody core structure (IgA, IgD, IgE, IgG or IgM). Examples of this kind of multi-specific molecules are described in Mertens, N., et. al., J. Immunol. (2000) 165(12): 7050-7; Holliger, P., et. al, (2005) Nature Biotech 23, 1126-1136; Fischer, N., and Leger, O., (2007) Pathobiology 74 3-14; Shen, J., et al., J. Immunol. Methods 318 (2007) 65-74; Wu, C., et al., and Nature Biotech 25 (2007) 1290-1297.

Alternatively, another kind of the multi-specific formats retains the antibody core structure resembling an immunoglobulin and contains CH2 and/or CH3 domains. This type of multi-specific formats is disclosed in a number of patents or published patent applications, for example, U.S. Pat. No. 4,444,878; U.S. Pat. No. 5,959,083; and U.S. Pat. No. 7,612,181.

Despite the wealth of the formats of multi-specific molecules developed so far, it is of great significance to develop novel formats with desired binding specificities and physical properties.

### BRIEF SUMMARY OF THE INVENTION

In one embodiment, the present invention provides a molecule comprising a first antibody having two light chains and two heavy chains with each of the two heavy chains comprising a variable region and a constant region having a CH1 domain, hinge domain, a CH2 domain, and a CH3 domain comprising a N-terminus and a C-terminus, wherein the CH3 domain of one of said heavy chains is connected at its C-terminus to a heavy chain variable region (VH) of a second antibody, and that CH3 domain of the other heavy chain is connected at its C-terminus to a light chain variable region (VL) of said second antibody, wherein said VH and VL of said second antibody are separate peptides.

In one aspect, the VH or VL comprises a N-terminus and a C-terminus, and wherein the C-terminus of said VH is further connected with the N-terminus of a VH of said second antibody while the C-terminus of the VL is further connected with the N-terminus of a VL of said second antibody.

In another aspect, the molecule has dual or multiple binding specificities.

In a further aspect, the molecule specifically binds to two different antigens.

In another further aspect, the molecule specifically binds to two different epitopes of an antigen.

In yet another aspect, the molecule specifically binds to HER1 and HER2.

In yet another aspect, the molecule has substantially same Tm as said first or second antibody.

In yet another aspect, the molecule has substantially same yield as the first antibody when expressed in the same (or comparable) expression systems.

In yet another aspect, the molecule is modified to enhance heterodimerization of its two heavy chains.

In one further aspect, the molecule is modified by introducing into one of the CH3 domains one or more mutations selected from a group consisting of: Ser364Leu, Thr366Val, Leu368Gln, Asp399Lys, Phe405Ser, Lys409Phe and Thr411Lys.

In another further aspect, the molecule is further modified by introducing into the other CH3 domain one or more mutations selected from a group consisting of: Tyr407Phe, Lys409Gln and Thr411Asp.

In another embodiment, the present invention provides a molecule comprising a first antibody having one light chain and two heavy chains, wherein one of the two heavy chains includes a variable region and a constant region having a CH1 domain, hinge domain, a CH2 domain, and a CH3 domain, while the other heavy chain consists essentially of a hinge domain, a CH2 domain, and a CH3 domain, wherein the CH3 domain of one of said heavy chains is connected at its C-terminus to a heavy chain variable region (VH) of a second antibody, and the CH3 domain of the other heavy chain is connected at its C-terminus to a light chain variable region (VL) of said second antibody, wherein said VH and VL of said second antibody are separate peptides.

In yet another embodiment, the present invention provides a molecule comprising a pair of antibody fragments, each of which consisting of a CH2 domain connected at its C-terminus with a CH3 domain (CH2-CH3), wherein one CH2 domain is connected at its N-terminus to a heavy chain variable region (VH) of a first antibody, and the other CH2 domain is connected at its N-terminus to a light chain variable region (VL) of said first antibody; and wherein one CH3 domain is connected at its C-terminus to a heavy chain variable region (VH) of a second antibody, and the other CH3 domain is connected at its C-terminus to a light chain variable region (VL) of said second antibody.

In another embodiment, the present invention includes a method of producing the molecule of the present invention, comprising: producing a DNA construct comprising a nucleic acid molecule encoding a heavy chain of said first antibody connected to a nucleic acid molecule encoding a) a VL, b) a tandem of VL-VL, c) a VH or d) a tandem of VH-VH of said second antibody; introducing said DNA construct into an expression vector; co-transfecting said expression vector into a host cell together with an expression vector comprising a nucleic acid molecule encoding the light chain of said first antibody; and expressing and assembling the molecule in said host cell.

In yet another embodiment, the present invention includes a method of enhancing hetero-dimerization of two heterologous polypeptides, each of which comprises a CH3 domain of an IgG, comprising introducing into one CH3 domain one or more mutations selected from a group consisting of: Ser364Leu, Thr366Val, Leu368Gln, Asp399Lys, Phe405Ser, Lys409Phe and Thr411Lys, and/or introducing into the other CH3 domain one or more mutations selected from a group consisting of: Tyr407Phe, Lys409Gln and Thr411Asp.

In one embodiment, the present invention provides a molecule comprising a first antibody having two light chains and two heavy chains with each of the two heavy chains comprising a variable region and a constant region having a CH1 domain, hinge domain, a CH2 domain, and a CH3 domain comprising a N-terminus and a C-terminus, wherein the CH3 domain of one of said heavy chains is connected at its C-terminus to one or more heavy chain variable region (VH) of a second antibody, and that CH3 domain of the other heavy chain is connected at its C-terminus to one or more light chain variable region (VL) of said second antibody, wherein said VH and VL of said second antibody are separate peptides; and wherein said CH3 domain contains one or more mutations selected from a group consisting of: Ser364Leu, Thr366Val, Leu368Gln, Asp399Lys, Phe405Ser, Lys409Phe and Thr411Lys, and/or introducing into the other CH3 domain one or more mutations selected from a group consisting of: Tyr407Phe, Lys409Gln and Thr411Asp.

In another embodiment, the present invention is directed to a molecule comprising a first antibody having one light chain and two heavy chains, wherein one of the two heavy chains includes a variable region and a constant region having a CH1 domain, hinge domain, a CH2 domain, and a CH3 domain, while the other heavy chain consists essentially of a hinge domain, a CH2 domain, and a CH3 domain, wherein the CH3 domain of one of said heavy chains is connected at its C-terminus to a heavy chain variable region (VH) of a second antibody, and the CH3 domain of the other heavy chain is connected at its C-terminus to a light chain variable region (VL) of said second antibody, wherein said VH and VL of said second antibody are separate peptides; and wherein said CH3 domain contains one or more mutations selected from a group consisting of: Ser364Leu, Thr366Val, Leu368Gln, Asp399Lys, Phe405Ser, Lys409Phe and Thr411Lys, and/or introducing into the other CH3 domain one or more mutations selected from a group consisting of: Tyr407Phe, Lys409Gln and Thr411Asp.

In yet another embodiment, the present invention provides a molecule comprising a pair of antibody fragments, each of which consisting of a CH2 domain connected at its C-terminus with a CH3 domain (CH2-CH3), wherein one CH2 domain is connected at its N-terminus to a heavy chain variable region (VH) of a first antibody, and the other CH2 domain is connected at its N-terminus to a light chain variable region (VL) of said first antibody; and wherein one CH3 domain is connected at its C-terminus to a heavy chain variable region (VH) of a second antibody, and the other CH3 domain is connected at its C-terminus to a light chain variable region (VL) of said second antibody; and wherein said CH3 domain contains one or more mutations selected from a group consisting of: Ser364Leu, Thr366Val, Leu368Gln, Asp399Lys, Phe405Ser, Lys409Phe and Thr411Lys, and/or introducing into the other CH3 domain one or more mutations selected from a group consisting of: Tyr407Phe, Lys409Gln and Thr411Asp.

In one embodiment, the present invention provides a molecule comprising a Fab region of a first antibody, wherein the CH1 domain of said Fab region connects at its C-terminus with at least the first 5, at least the first 6, at least the first 7, at least the first 8, at least the first 9, at least the first 10, or at least the first 15 amino acids of the hinge region of said first antibody, which in turn connects to a VH region of a second antibody via a first linker, and the CL domain of said Fab region connects at its C-terminus a VL region of said second antibody via a second linker. The linkers are designed such that the space between CH1 and VH of the second antibody is the same as the space between CL and VL of the second antibody. In one embodiment, the first linker is three (3) G₄S, and the second linker is four (4) G₄S.

In yet another embodiment, the present invention includes a multispecific and/or multivalent molecules described herein for use as a medication in treating cancer, autoimmune diseases, and/or infectious diseases. The present invention also includes use of the molecules described herein in treating cancer, autoimmune disease, and/or infectious diseases.

### BRIEF DESCRIPTION OF THE DRAWINGS

FIG. 1 shows the design for the multi-valent and/or multi-specific molecules of the present invention.

FIG. 2 shows the bispecific molecules of the present invention are expressed at similar level as the corresponding parental antibody.

FIG. 3 shows expression of one arm molecule by SDS-PAGE.

FIG. 4 shows expression of one arm molecule by Western blot using anti-Kappa antibody.

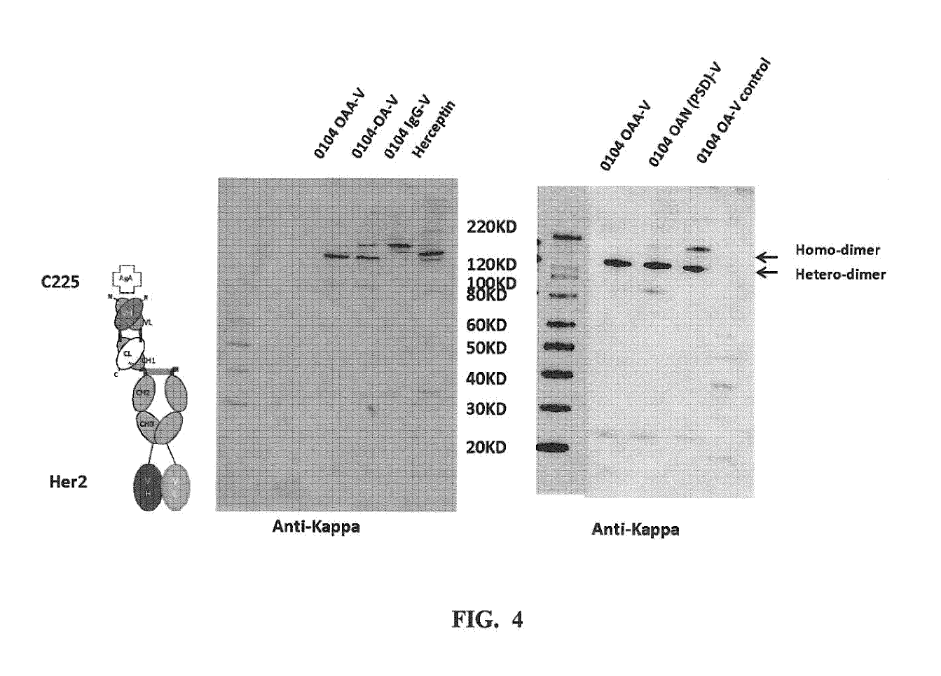

FIG. 5 shows dual binding activity of the produced bispecific molecule demonstrated by cross-link ELISA or dual ligand binding analysis.

FIG. 6 shows ELISA-bi-specific binding of one arm molecules and EC50 determination.

FIG. 7 shows the thermostability analysis by DSF indicating that the produced bispecific molecules have similar Tm as compared to that of C225.

FIG. 8 shows the binding activity of the bispecific molecules (1E format) with different linkers.

FIG. 9 shows expression of the bispecific molecules (1E format) by SDS-PAGE.

FIG. 10 shows dual binding activity of the bispecific molecules (1E format).

### DETAILED DESCRIPTION OF THE INVENTION

### Definitions

In order that the present invention may be more readily understood, certain terms are first defined. Additional definitions are set forth throughout the detailed description. Unless defined otherwise, all technical and scientific terms used herein have the same meaning as commonly understood by those of ordinary skill in the art to which this invention pertains.

The term “antibody” as used herein refers to whole antibodies that interact with (e.g., by binding, steric hinderance, stabilizing/destabilizing, spatial distribution) an antigenic epitope. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelised antibodies, chimeric antibodies, bi-specific or multiple-specific antibody and anti-idiotypic (anti-Id) antibodies (including, e.g., anti-Id antibodies to antibodies of the invention). The antibodies can be of any isotype (e.g., IgG, IgE, IgM, IgD, IgA and IgY), class (e.g., IgG1, IgG2, IgG3, IgG4, IgA1 and IgA2) or subclass.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively.

The term “antibody fragment” as used herein refers to one or more portions of an antibody. In some embodiments, these portions are part of the contact domain(s) of an antibody. In some other embodiments, these portion(s) are antigen-binding fragments that retain the ability to specifically interact with (e.g., by binding, steric hinderance, stabilizing/destabilizing, spatial distribution) an epitope. Examples of binding fragments include, but are not limited to, single-chain Fvs (scFv), a Fab fragment, a monovalent fragment consisting of the VL, VH, CL and CH1 domains; a F(ab)₂ fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR).

Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23: 1126-1136). Antibody fragments can be grafted into scaffolds based on polypeptides such as Fibronectin type III (Fn3) (see U.S. Pat. No. 6,703,199, which describes fibronectin polypeptide monobodies).

Antibody fragments can be incorporated into single chain molecules comprising a pair of tandem Fv segments (VH-CH1-VH-CH1) which, together with complementary light chain polypeptides, form a pair of antigen binding regions (Zapata et al., (1995) Protein Eng. 8:1057-1062; and U.S. Pat. No. 5,641,870).

The term “antibody-like molecule” refers to a molecule comprising an antibody or a fragment thereof.

The term “single-chain Fv” or “scFv” as used herein refers to antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Plückthun in The Pharmacology of Monoclonal Antibodies, vol. 113, Rosenburg and Moore eds., (1994) Springer-Verlag, New York, pp. 269-315.

The term “diabody” as used herein refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., (1993) Proc. Natl. Acad. Sci. USA 90:6444-6448.

The term “mono-specific molecule,” as used herein, refers to a molecule that binds to one epitope on a target antigen. In some embodiments, a mono-specific molecule of the present invention is a mono-specific antibody like molecule. In some embodiments, a mono-specific molecule of the present invention is a mono-specific antibody.

The term “bi-specific molecule” as used herein refers to a molecule that binds to two different epitopes on one antigen or two different antigens. In some embodiments, a bi-specific molecule of the present invention is a bi-specific antibody like molecule. In some embodiments, a bi-specific molecule of the present invention is a bi-specific antibody.

The term “multi-specific molecule” as used herein refers to a molecule that binds to two or more different epitopes on one antigen or on two or more different antigens. In some embodiments, a multi-specific molecule of the present invention is a multi-specific antibody like molecule. In some embodiments, a multi-specific molecule of the present invention is a multi-specific antibody. The term “multi-specific” includes “bi-specific.”

The term “a tandem of VH domains (or VHs)” as used herein refers to a string of VH domains, consisting of multiple numbers of identical VH domains of an antibody. Each of the VH domains, except the last one at the end of the tandem, has its C-terminus connected to the N-terminus of another VH domain with or without a linker. A tandem has at least 2, 3, 4, 5, 6, 7, 8, 9, 10, 11, 12, 13, 14, 15, 16, 17, 18, 19, 20, 25, 30, 50, or 100 VH domains. The tandem of VH can be produced by joining the encoding genes of each VH domain in a desired order using recombinant methods with or without a linker (e.g., a synthetic linker) that enables them to be made as a single protein. In one aspect, the VH domains in the tandem, alone or in combination with VL domains of the same antibody, retain the binding specificity of the original antibody. The N-terminus of the first VH domain in the tandem is defined as the N-terminus of the tandem, while the C-terminus of the last VH domain in the tandem is defined as the C-terminus of the tandem.

The term “a tandem of VL domains (or VLs)” as used herein refers to a string of VL domains, consisting of multiple numbers of identical VL domains of an antibody. Each of the VL domains, except the last one at the end of the tandem, has its C-terminus connected to the N-terminus of another VH with or without a linker. A tandem has at least 2, 3, 4, 5, 6, 7, 8, 9, 10, 11, 12, 13, 14, 15, 16, 17, 18, 19, 20, 25, 30, 50, or 100 VL domains. The tandem of VL can be produced by joining the encoding gene of each VL domain in a desired order using recombinant methods with or without a linker (e.g., a synthetic linker) that enables them to be made as a single protein. Preferably, the VL domains in the tandem, alone or in combination with VH domains of the same antibody, retain the binding specificity of the original antibodies. The N-terminus of the first VL domain in the tandem is defined as the N-terminus of the tandem, while the C-terminus of the last VL domain in the tandem is defined as the C-terminus of the tandem.

The term “monovalent molecule” as used herein refers to a molecule that has a single antigen binding site. In some embodiments, a monovalent molecule of the present invention is a monovalent antibody like molecule. In some embodiments, a monovalent molecule of the present invention is a monovalent antibody.

The term “bivalent molecule” as used herein refers to a molecule that has two antigen binding sites. In some embodiments, a bivalent molecule of the present invention is a bivalent antibody like molecule. In some embodiments, a bivalent molecule of the present invention is a bivalent antibody.

The term “trivalent molecule” as used herein refers to a molecule that has three antigen binding sites. In some embodiments, a trivalent molecule of the present invention is a trivalent antibody like molecule. In some embodiments, a trivalent molecule of the present invention is a trivalent antibody.

The term “tetravalent molecule” as used herein refers to a molecule that has four antigen binding sites. In some embodiments, a tetravalent molecule of the present invention is a tetravalent antibody like molecule. In some embodiments, a tetravalent molecule of the present invention is a tetravalent antibody.

The term “multivalent molecule” as used herein refers to a molecule that has at least two antigen binding sites. In some embodiments, a multivalent molecule of the present invention is a multivalent antibody like molecule. In some embodiments, a multivalent molecule of the present invention is a multivalent antibody. In some embodiments, a multivalent molecule is a bivalent molecule, trivalent molecule or a tetravalent molecule.

The term “substantially similar,” or “substantially the same,” as used herein refers to a sufficiently high degree of similarity between two numeric values (generally one associated with an antibody-like molecule of the invention and the other associated with a reference/comparator antibody or antibody-like molecule) such that one of skill in the art would consider the difference between the two values to be of little or no biological and/or statistical significance within the context of the biological characteristic measured by said values (e.g., Tm values or the amount of the assembled antibodies). The difference between said two values is preferably less than about 50%, preferably less than about 40%, preferably less than about 30%, preferably less than about 20%, preferably less than about 10% as a function of the value for the reference/comparator antibody.

The term “substantially same yield” as used herein refers to the amount of an assembled molecule (e.g. antibody or antibody-like molecule) of the present invention is substantially the same as that of the antibodies it derived from when being prepared under similar conditions in similar cell types. Relative yields of antibody or antibody like products can be determined using standard methods including scanning densitometry of SDS-PAGE gels and/or immunoblots and the AME5-RP assay.

The term “thermostability” as used herein refers to the ability of a protein (e.g., an antibody or an antibody-like molecule) to retain the characteristic property when heated moderately. When exposed to heat, proteins will experience denaturing/unfolding process and will expose hydrophobic residues. A protein is completely unfolded in response to heat at a characteristic temperature. The temperature at the mid-point of the protein unfolding process is defined as Tm, which is an important physical characteristic of a protein, and can be measured with the techniques known in the art, such as by monitoring the denaturing process using Sypro orange dye labeling hydrophobic residues of denatured proteins or by using differential scanning calorimetry (DSC) techniques.

The term “epitope” as used herein refers to any determinant capable of binding with high affinity to an antibody or an antibody-like molcule. An epitope is a region of an antigen that is bound by an antibody (or an antibody-like molecule) that specifically targets that antigen, and when the antigen is a protein, includes specific amino acids that directly contact the antibody or the antibody-like molecule. Most often, epitopes reside on proteins, but in some instances, may reside on other kinds of molecules, such as nucleic acids. Epitope determinants may include chemically active surface groupings of molecules such as amino acids, sugar side chains, phosphoryl or sulfonyl groups, and may have specific three dimensional structural characteristics, and/or specific charge characteristics.

Generally, antibodies or antibody-like molecules specific for a particular target antigen will preferentially recognize an epitope on the target antigen in a complex mixture of proteins and/or macromolecules.

Regions of a given polypeptide that include an epitope can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., (1984) Proc. Natl. Acad. Sci. USA 8:3998-4002; Geysen et al., (1985) Proc. Natl. Acad. Sci. USA 82:78-182; Geysen et al., (1986) Mol. Immunol. 23:709-715. Similarly, conformational epitopes are readily identified by determining spatial conformation of amino acids such as by, e.g., x-ray crystallography and two-dimensional nuclear magnetic resonance. See, e.g., Epitope Mapping Protocols, supra. Antigenic regions of proteins can also be identified using standard antigenicity and hydropathy plots, such as those calculated using, e.g., the Omiga version 1.0 software program available from the Oxford Molecular Group. This computer program employs the Hopp/Woods method, Hopp et al., (1981) Proc. Natl. Acad. Sci USA 78:3824-3828; for determining antigenicity profiles, and the Kyte-Doolittle technique, Kyte et al., (1982) J. Mol. Biol. 157:105-132; for hydropathy plots.

Specific binding between two entities means a binding with an equilibrium constant (K_{A}) (kₒₙ/k_{off}) of at least 10²M⁻¹, at least 5×10²M⁻¹, at least 10³M⁻¹, at least 5×10³M⁻¹, at least 10⁴M⁻¹ at least 5×10⁴M⁻¹, at least 10⁵M⁻¹, at least 5×10⁵M⁻¹, at least 10⁶M⁻¹, at least 5×10⁶M⁻¹, at least 10⁷M⁻¹, at least 5×10⁷M⁻¹, at least 10⁸M⁻¹, at least 5×10⁸M⁻¹, at least 10⁹M⁻¹, at least 5×10⁹M⁻¹, at least 10¹° M⁻¹, at least 5×10¹⁰M⁻¹ at least 10¹¹M⁻¹, at least 5×10¹¹M⁻¹, at least 10¹²M⁻¹, at least 5×10¹²M⁻¹, at least 10¹³M⁻¹, at least 5×10¹³M⁻¹, at least 10¹⁴M⁻¹, at least 5×10¹⁴M⁻¹, at least 10¹⁵M⁻¹, or at least 5×10¹⁵M⁻¹.

The term “specifically (or selectively) binds” to an antigen or an epitope refers to a binding reaction that is determinative of the presence of a cognate antigen or an epitope in a heterogeneous population of proteins and other biologics. In addition to the equilibrium constant (K_{A}) noted above, an antibody or antibody-like molecule of the invention typically also has a dissociation rate constant (K_{D}) (k_{off}/kₒₙ) of less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, less than 10⁻⁹M, less than 5×10⁻¹⁰M, less than 10⁻¹⁰M, less than 5×10⁻¹¹M, less than 10⁻¹¹M, less than 5×10⁻¹²M, less than 10⁻¹²M, less than 5×10⁻¹³M, less than 10⁻¹³M, less than 5×10⁻¹⁴M, less than 10⁻¹⁴M, less than 5×10⁻¹⁵M, or less than 10⁻¹⁵M or lower, and binds to the target antigen with an affinity that is at least two-fold greater than its affinity for binding to a non-specific antigen (e.g., HSA).

The terms “a molecule (e.g. an antibody or an antibody-like molecule) recognizing an antigen (or an epitope)” and “a molecule specific for an antigen (or an epitope)” are used interchangeably herein with the term “a molecule which binds specifically to an antigen (or an epitope)”. In one embodiment, the molecule (e.g. antibody or antibody-like molecule) or fragment thereof has dissociation constant (K_{d}) of less than 3000 pM, less than 2500 pM, less than 2000 pM, less than 1500 pM, less than 1000 pM, less than 750 pM, less than 500 pM, less than 250 pM, less than 200 pM, less than 150 pM, less than 100 pM, less than 75 pM, less than 10 pM, less than 1 pM as assessed using a method described herein or known to one of skill in the art (e.g., a BIAcore assay, ELISA, FACS, SET) (Biacore International AB, Uppsala, Sweden). The term “K_{assoc}” or “Kₐ”, as used herein, refers to the association rate of a particular antibody-antigen interaction, whereas the term “K_{dis}” or “K_{d},” as used herein, refers to the dissociation rate of a particular antibody-antigen interaction. The term “K_{D}”, as used herein, refers to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of an antibody is by using surface plasmon resonance, or using a biosensor system such as a Biacore® system.

The term “multiple binding specificities” as used herein refers to that a molecule of the present invention (e.g., an antibody or an antibody like molecule) is capable of specifically binding at least two, three, four, five, six, seven, eight, nine, or ten different epitopes either on the same antigen or on at least two, three, four, five, seven, eight, nine or ten different antigens.

The term “dual binding specificity” as used herein refers to that a molecule of the present invention (e.g., an antibody or an antibody like molecule) is capable of binding two different epitopes either on the same antigen or on two different antigens.

The term “isolated antibody” or “isolated antibody-like molecule” as used herein refers to an antibody or an antibody-like molecule that is substantially free of other antibodies or antibody-like molecules having different antigenic specificities. Moreover, an isolated antibody or antibody-like molecule may be substantially free of other cellular material and/or chemicals.

The term “monoclonal antibody” or “monoclonal antibody composition” as used herein refers to polypeptides, including antibodies, antibody fragments, molecules, etc. that have substantially identical to amino acid sequence or are derived from the same genetic source. This term also includes preparations of antibody molecules of single molecular composition. A monoclonal antibody composition displays a single binding specificity and affinity for a particular epitope.

The term “humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin lo sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1: 105-115 (1998); Harris, Biochem. Soc. Transactions 23:1035-1038 (1995); Hurle and Gross, Curr. Op. Biotech. 5:428-433 (1994).

The term “human antibody” as used herein includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik, et al. (2000. J Mol Biol 296, 57-86). The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Al Lazikani et al., (1997) J. Mol. Bio. 273:927 948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mol. Biol. 273:927-948.

The human antibodies of the invention may include amino acid residues not encoded by human sequences (e.g., mutations introduced by random or site-specific mutagenesis in vitro or by somatic mutation in vivo, or a conservative substitution to promote stability or manufacturing). However, the term “human antibody”, as used herein, is not intended to include antibodies in which CDR sequences derived from the germline of another mammalian species, such as a mouse, have been grafted onto human framework sequences.

A “modification” of an amino acid residue/position, as used herein, refers to a change of a primary amino acid sequence as compared to a starting amino acid sequence, wherein the change results from a sequence alteration involving said amino acid residue/positions. For example, typical modifications include substitution of the residue (or at said position) with another amino acid (e.g., a conservative or non-conservative substitution), insertion of one or more amino acids adjacent to said residue/position, and deletion of said residue/position. An “amino acid substitution,” or variation thereof, refers to the replacement of an existing amino acid residue in a predetermined (starting) amino acid sequence with a different amino acid residue. Generally and preferably, the modification results in alteration in at least one physicobiochemical activity of the variant polypeptide compared to a polypeptide comprising the starting (or “wild type”) amino acid sequence. For example, in the case of an antibody, a physicobiochemical activity that is altered can be binding affinity, binding capability and/or binding effect upon a target molecule.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some embodiments, the phrase “conservative sequence modifications” are used to refer to amino acid modifications that do not significantly affect or alter the binding characteristics of the antibody or the antibody-like molecule containing the amino acid sequence.

The terms “percent identical” or “percent identity,” in the context of two or more nucleic acids or polypeptide sequences, refers to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

The term “comparison window” as used herein includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller (Comput. Appl. Biosci. 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453 (1970)) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., (1991) Nucleic Acid Res. 19:5081; Ohtsuka et al., (1985) J. Biol. Chem. 260:2605-2608; and Rossolini et al., (1994) Mol. Cell. Probes 8:91-98).

The term “operably linked” or functionally linked, as used herein, refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “polypeptide” and “protein” are used interchangeably herein to refer to a polymer of amino acid residues. The phrases also apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “in vivo half life”, as used herein, refers to the half-life of the molecule of interest or variants thereof circulating in the blood of a given mammal.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Various aspects of the invention are described in further detail in the following sections and subsections.

### I. Novel Formats for Multi-specific Molecules

### A. Structure

The present invention includes multi-specific or mono-specific molecules of various formats.

### Format 1

In one aspect, the present invention includes a molecule that is derived from a first antibody comprising two heavy (H) chains and two light (L) chains inter-connected by disulfide bonds. Each heavy chain is comprised of a heavy chain variable region (abbreviated herein as VH) and a heavy chain constant region. The heavy chain constant region is comprised of CH1 domain, hinge domain, CH2 domain and CH3 domain.

This molecule comprises the first antibody with one CH3 domain of the heavy chain connected in its C-terminus to one heavy chain variable region (VH) or a tandem of the heavy chain variable regions of a second patent antibody; and the other CH3 domain of the other heavy chain connected in its C-terminus to one light chain variable region (VL) or a tandem of light chain variable regions of a second antibody. The VH and VL of the second antibody may have the same or different binding specificity from that of the first antibody. Therefore, format 1 comprises a hybrid heavy chain comprising a heavy chain of the first antibody connected at its C-terminus with a VH domain (or a tandem thereof) or a VL domain (or a tandem thereof) of the second antibody. In some embodiments, the C-terminus of the CH3 domain of the first antibody is connected with the N-terminus of the VH (or a tandem thereof) or VL (or a tandem thereof) of the second antibody.

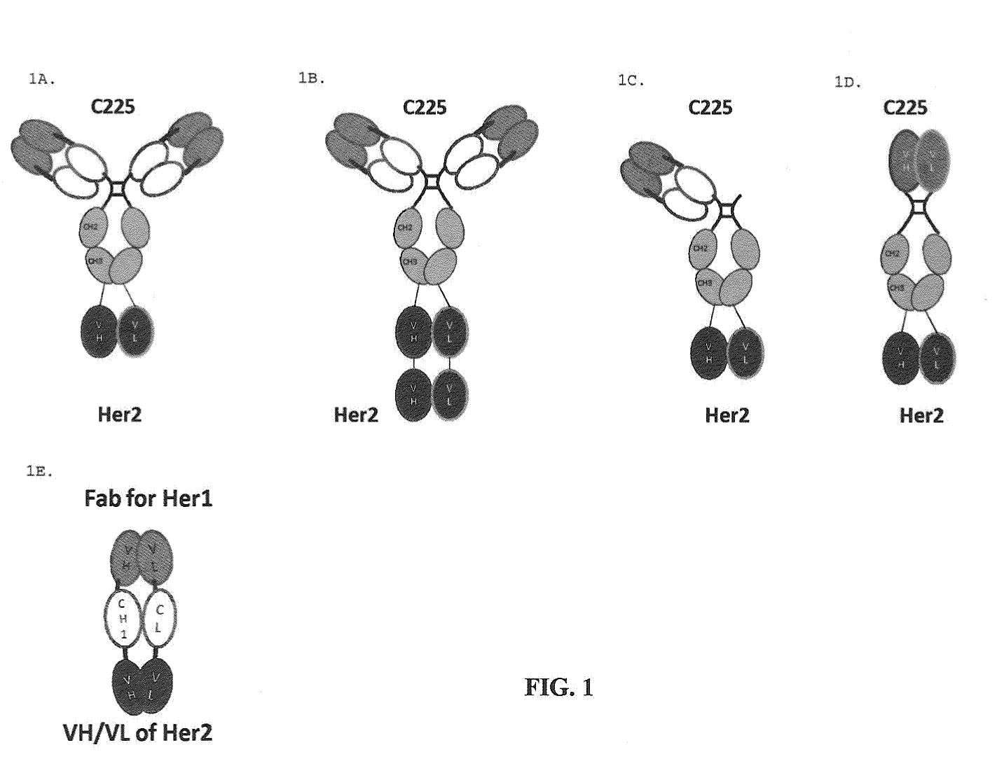

In one embodiment, one of the CH3 domains is connected with a single VH domain of the second antibody while the other CH3 domain is connected with a single VL domain of the second antibody. In another embodiment, one of the CH3 domains is connected with a tandem of VH domains from the second antibody while the other CH3 domain is connected with a tandem of VL domains from the second antibody. The VH tandem and VL tandem may contain same or different numbers of VH domains and VL domains respectively, but preferably, contain the same number of VH or VL domains. In an exemplary embodiment, the VH tandem and VL tandem consist of two VH domains (VH-VH) and two VL domains (VL-VL) respectively. Accordingly, a trivalent antibody (see FIGS. 1A and 1B) is formed with two N-terminal antigen binding sites, and with one C-terminal antigen binding site.

### Format 2

Format 2 is a variant “one-arm format” of Format 1. In this format, the first antibody is not a whole antibody and has at least a portion of one heavy chain and/or one light chain missing. In an exemplary embodiment, one heavy chain of the first antibody has the variable region missing. In a further exemplary embodiment, both variable region and the CH1 domain of one heavy chain are missing. As a result, the light chain that is inter-connected with the variable region of this heavy chain is also missing from the first antibody. Accordingly, in this embodiment, the first antibody only has one complete heavy chain, one light chain inter-connected with this heavy chain, and another partial heavy chain consisting essentially of the hinge, CH2 and CH3 domains (see FIG. 1C). Thus, the molecule has one N-terminal antigen-binding site and one C-terminal binding site.

### Format 3

Format 3 is another variant format of Format 1. As shown in FIG. 1D, in this format, the created molecule comprises a pair of antibody fragments, each of which consisting of a CH2 domain connected at its C-terminus with a CH3 domain (CH2-CH3). The CH2 and CH3 are derived from a constant region of an antibody described herein. In some embodiments, each CH3 domain of this pair of CH2-CH3 fragments is dimerized with each other. In one particular embodiment, one CH2 domain of the CH2-CH3 pair is connected at its N-terminus to the C-terminus of a heavy chain variable region (VH) of a first antibody, and the other CH2 domain is connected at its N-terminus to the C-terminus of a light chain variable region (VL) of said first antibody. In addition, one CH3 domain of this CH2-CH3 pair is connected at its C-terminus to the N-terminus of a heavy chain variable region (VH) of a second antibody, and the other CH3 domain is connected at its C-terminus to the N-terminus of a light chain variable region (VL) of said second antibody. The first and second antibody may have the same or different binding specificity.

In some embodiments, the VH (or tandem of VH) and VL (or tandem of VL) at the C-terminus of the molecules with Format 1, 2, or 3, are separate peptides and are not directly connected artificially between each other to form a single peptide, for example, using recombinant technology or chemical coupling or non-covalent association, etc. In one particular aspect, they are not connected in such a way to form a scFv or a diabody. For example, the VH(s) and VL(s) are not linked by a synthetic linker that enables them to be made as a single protein chain in which the VL and VH regions pair to form monovalent molecules (see e.g., Bird et al., (1988) Science 242:423-426; and Huston et al., (1988) Proc. Natl. Acad. Sci. 85:5879-5883). Nevertheless, the VH (or tandem of VH) and VL (or tandem of VL) at the C-terminus of the molecule can be re-associated between each other to form an antigen binding site. In one particular aspect, the VH or VL is not a single domain antibody that is capable of binding to target antigen by itself, and association between VH and VL is required for retaining antigen binding capability/specificity.

In other embodiments, the VH or VL at the C-terminus of the molecules of Format 2 or the VH or VL at the N-terminus and C-terminus of the molecules of Format 3 can be a single domain antibody that is capable of binding to target antigen by itself.

To produce the molecules of Formats 1 or 2 of the present invention, the C-terminus of the CH3 of the first antibody is functionally linked (e.g., by chemical coupling, genetic fusion, non-covalent association or otherwise) to the N-terminus of VH or VL or a tandem thereof of the second antibody. In addition, to produce the molecules of Format 3, the C-terminus of CH3 and the N-terminus of CH2 needs to be functionally linked to the desired VH or VL domains. In an exemplary embodiment, these CH3 (or CH2) domains are connected with the desired VH(s) or (VL) through a peptide linker. In one embodiment, the peptide linker comprises an amino acid sequence of GGGGS (SEQ ID NO. 21) (Glycine-Glycine-Glycine-Glycine-Serine, also referred as “G₄5”). In a further embodiment, the peptide linker comprises at least 1, 2, 3, 4, 5, 6, 7, 8, 9, 10, 11, 12, 15, or 20 G₄5. In an exemplary embodiment, the peptide linker comprises 0-4 G₄5. Other peptide linkers available in the art are also included in the present invention.

### Format 4

Format 4 describes a molecule comprising a Fab region of a first antibody, wherein the CH1 domain of said Fab region connects at its C-terminus with at least the first 5, at least the first 6, at least the first 7, at least the first 8, at least the first 9, at least the first 10, or at least the first 15 amino acids of the hinge region of said first antibody, which in turn connects to a VH region of a second antibody via a first linker, and the CL domain of said Fab region connects at its C-terminus a VL region of said second antibody via a second linker. The first and second antibody may have the same or different binding specificity. An example of the format 4 molecules is shown in FIG. 1E.

The linker used in this format can be a linker known in the art. The linkers are designed such that the space between CH1 and VH of the second antibody is the same as the space between CL and VL of the second antibody. In one embodiment, the first linker is three (3) G₄S, and the second linker is four (4) G₄S.

Format 4 molecules include the upper hinge region of the first antibody with one cysteine retained. For example, for an IgG1 human antibody, the sequence is EPKSCDKTHT.

### B. Binding Specificity

The molecules of the present invention can be multivalent multi-specific, multivalent mono-specific, monovalent multi specific, or monovalent monospecific.

In one embodiment, the molecule is a bivalent molecule (e.g., a bivalent antibody like molecule) with one N-terminal binding region and one C-terminal antigen binding region. In one aspect, the molecule has a structure of Format 2, where the first antibody only has one N-terminal binding site. In another aspect, the molecule has a structure of Format 3. In one particular aspect, the present molecule has dual binding specificities if the first antibody and second antibody recognize two different antigens or two different epitopes on the same antigen. In another particular aspect, if the first and second antibodies bind to the same antigen/epitope, the molecule is a mono-specific molecule having the same binding specificity of the first and second antibodies.

In another embodiment, the molecule of the present invention is a trivalent molecule (e.g. a trivalent antibody-like molecule) of Format 1 having two N-terminal binding sites derived from the first antibody and one C-terminal binding site derived from the second antibody. In one aspect, the first and second antibodies bind to the same epitope or the same antigen. As a result, the present molecule is a trivalent mono-specific antibody-like molecule. In another aspect, the first antibody and second antibodies recognize two different antigens, or two different epitopes on the same antigen. The present molecule is thereby a bi-specific antibody-like molecule having binding specificity the two different antigens, or the two different epitopes. In yet another aspect, the first antibody is a bi-specific antibody itself and recognizes two different epitopes or antigens. If the second antibody binds to an epitope or antigen that is same as one of the two epitopes or antigens bound by the first antibody, the generated molecule has dual binding specificity with two N-terminal binding sites recognizing two different antigens/epitopes, while the C-terminal binding site recognizing one of the two antigens/epitopes. If the second antibody binds to an epitope or antigen that is different from the two epitopes or antigens bound by the first antibody, the generated molecule has tri-specificities with the two N-terminal binding site recognizing two different antigens/epitopes, while the C-terminal binding site recognizing another different antigens/epitopes. The three different epitopes may be on the same antigen or two or three different antigens.

Standard assays to evaluate the binding specificity of the antibodies or antibody-like molecules toward various epitopes and/or antigens are known in the art, including for example, Biacore analysis, or FACS relative affinity (Scatchard), ELISAs, western blots and RIAs. Suitable assays are described in detail in the Definition and Examples.

### C. Physical Property/Yield

In certain embodiments, the present molecule offers desirable physical properties, such as a thermo-stability substantially same as that of the first and/or second antibodies.

Thermostability refers to protein stability during heat stress, which is an ability of a protein to retain the characteristic property when heated moderately. When exposed to heat, proteins will experience denaturing/unfolding process and will expose hydrophobic residues. Each protein is completely unfolded in response to heat at a characteristic temperature. The temperature at the mid-point of the protein unfolding process is defined as Tm, which is an important physical characteristic for a protein, and can be measured with the techniques known in the art. A multi-specific molecule having a relatively high value of Tm is usually desirable because a high value of Tm often indicates less aggregation when it is used for preparing a pharmaceutical composition. In addition, higher Tm may also result in higher expression and yield.

In one particular embodiment, the multi-specific molecule of the present invention has substantially same Tm as compared to that of one of the parent antibodies (the first and second antibodies described herein). In one aspect, the present molecule has substantially same Tm as compared to that of the first antibody, which provides the multi-specific molecule with the antibody core structure.

In yet another embodiment, the present invention includes a method of generating a multi-specific molecule derived from at least one parent antibody and having substantially the same thermostability as the parent antibody comprising 1) designing a molecule of one of the formats described herein; 2) producing the molecule in a host cell; and 3) measuring and comparing Tm of the molecule and the parent antibody.

The molecules with the formats of the present invention have improved thermostability as compared to the molecules comprising scFv fusion to C-terminal CH3 of an antibody. The formats of the present invention avoid the low stability of scFv when present in an antibody-like molecule.

Cell culture systems have been widely used for expressing antibody fragments, but there have been few attempts to express and recover functional completely assembled full-length antibodies in high yield. Because of the complex structure and large size of completely assembled full-length antibodies, it is often difficult to achieve proper folding and assembly of the expressed heavy and light chains, especially in bacterial cells. This problem is especially challenging when producing multi-specific molecules. Because of the random pairing of two different antibody heavy and light chains within the host cells, only small percentage of the assembled antibody species is the desired, functional multi-specific molecules. Due to the presence of mispaired byproducts, and significantly reduced production yields, sophisticated purification procedures are required (see e.g. Morrison, S. L., Nature Biotech 25 (2007) 1233-1234).

In some embodiments, the present molecules (e.g. antibodies or antibody-like molecules), when recombinantly produced in comparable cell cultures, have substantially same yield as at least one of the parent antibodies (e.g. first or second antibody described herein), or as both parent antibodies. In particular, under the same culture condition, being expressed by the same type of host cells, the molecules have substantially the same expression levels as one or both parent antibodies. The expression levels of the produced molecule can be measured with the standard techniques in the art, such as, scanning densitometry of SDS-PAGE gels and/or immunoblots and the AME5-RP assay. Antibody yield can also be quantified by protein A sensor chip using Qctec Red (Fotrte Bio) as described in the Example.

The present invention includes a method of generating a molecule derived from at least one parent antibody and having substantially same yield as the parent antibody comprising 1) designing a molecule of the present invention described herein; 2) producing the molecule in a host cell; and 3) measuring and comparing the expression level of said molecule with said parent antibody.

### D. Modification of the Molecules of the Present Invention

1. Molecules with Enhanced Heterodimerization

Inadequate heterodimerization of two antibody heavy chains has always been an obstacle for increasing the yield of desired multi-specific molecules and represents challenges for purification. A variety of approaches available in the art can be used in for enhancing dimerization of the two heavy chains of bi-specific or multi-specific antibody-like molecule, as disclosed in EP 1870459A1; U.S. Pat. No. 5,582,996; U.S. Pat. No. 5,731,168; U.S. Pat. No. 5,910,573; U.S. Pat. No. 5,932,448; U.S. Pat. No. 6,833,441; U.S. Pat. No. 7,183,076; U.S. Patent Application Publication No. 2006204493A1; and PCT Publication No. WO2009/089004A1

The present invention provides methods of enhancing dimerization (hetero-dimerization) of two interacting heterologous polypeptides and/or reducing dimerization (homo-dimerization) of two identical polypeptides. Typically, each of the two interacting polypeptides comprises a CH3 domain of an antibody. The CH3 domains are derived from the constant region of an antibody of any isotype, class or subclass, and preferably of IgG (IgG1, IgG2, IgG3 and IgG4) class.

Typically, the polypeptides comprise other antibody fragments in addition to CH3 domains, such as, CH1 domains, CH2 domains, hinge domain, VH domain(s), VL domain(s), CDR(s), and/or antigen-binding fragments described herein. These antibody fragments are derived from various types of antibodies described herein, for example, polyclonal antibody, monoclonal antibodies, chimeric antibodies, humanized antibodies, human antibodies, bi-specific or multi-specific antibodies, camelised antibodies, anti-idiotypic (anti-Id) antibodies and antibody conjugates. In some embodiments, the two hetero-polypeptides are two heavy chains forming a bispecific or multi-specific molecules. Heterodimerzation of the two different heavy chains at CH3 domains give rise to the desired antibody or antibody-like molecule, while homodimerization of identical heavy chains will reduce yield of the desired antibody or molecule. In an exemplary embodiment, the two hetero-polypeptide chains are the two chains comprising CH3 domains and forming the molecules of Format 1, 2 and/or 3 of the present invention.

The method of the present invention comprises introducing amino acid mutations into the CH3 domains of the two interacting hetero-polypeptides. These mutations are designed based on the “polar-bridging” rational, which is to make residues at the binding interface of the two polypeptide chains to interact with residues of similar physical property in the heterodimer configuration, while with residues of different physical property in the homodimer configuration. In particular, these mutations are designed so that, in the heterodimer formation, polar residues interact with polar residues, while hydrophobic residues interact with hydrophobic residues. In contrast, in the homodimer formation, residues are mutated so that polar residues interact with hydrophobic residues. The favorable interactions in the heterodimer configuration and the unfavorable interactions in the homodimer configuration work together to make it more likely for CH3 domains to form heterodimers than to form homodimers.

In an exemplary embodiment, the above mutations are generated at one or more positions of residues 364, 368, 399, 405, 409, and 411 of CH3 domain, amino acid numbering according to the EU numbering scheme of Kabat et al. (pp. 688-696 in Sequences of proteins of immunological interest, 5th ed., Vol. 1 (1991; NIH, Bethesda, Md.)).

In one aspect, one or more mutations selected from a group consisting of: Ser364Leu, Thr366Val, Leu368Gln, Asp399Lys, Phe405Ser, Lys409Phe and Thr411Lys are introduced into one of the two CH3 domains. (Ser364Leu: original residue of serine at position 364 is replaced by leucine; Thr366Val: original residue of threonine at position 366 is replaced by valine; Leu368Gln: original residue of leucine at position 368 is replaced by glutamine;

Asp399Lys: original residue aspartic acid at position 399 is replaced by lysine; Phe405Ser: original residue phenylalanine at position 405 is replaced by serine; Lys409Phe: original residue lysine at position 409 is replaced by phenylalanine; Thr411Lys: original residue of threonine at position 411 is replaced by lysine.).

In another aspect, the other CH3 can be introduced with one or more mutations selected from a group consisting of: Tyr407Phe, Lys409Gln and Thr411Asp (Tyr407Phe: original residue tyrosine at position 407 is replaced by phenyalanine; Lys409Glu: original residue lysine at position 409 is replaced by glutamic acid; Thr411Asp: original residue of threonine at position 411 is replaced by aspartic acid).

In a further aspect, one CH3 domain has one or more mutations selected from a group consisting of: Ser364Leu, Thr366Val, Leu368Gln, Asp399Lys, Phe405Ser, Lys409Phe and Thr411Lys, while the other CH3 domain has one or more mutations selected from a group consisting of: Tyr407Phe, Lys409Gln and Thr411Asp.

In one exemplary embodiment, the original residue of threonine at position 366 of one CH3 domain is replaced by valine, while the original residue of tyrosine at position 407 of the other CH3 domain is replaced by phenylalanine.

In another exemplary embodiment, the original residue of serine at position 364 of one CH3 domain is replaced by leucine, while the original residue of leucine at position 368 of the same CH3 domain is replaced by glutamine.

In yet another exemplary embodiment, the original residue of phenylalanine at position 405 of one CH3 domain is replaced by serine and the original residue of lysine at position 409 of this CH3 domain is replaced by phenylalanine, while the original residue of lysine at position 409 of the other CH3 domain is replaced by glutamine.

In yet another exemplary embodiment, the original residue of aspartic acid at position 399 of one CH3 domain is replaced by lysine, and the original residue of threonine at position 411 of the same CH3 domain is replaced by lysine, while the original residue of threonine at position 411 of the other CH3 domain is replaced by aspartic acid.

The amino acid replacements described herein are introduced into the CH3 domains using techniques which are well known in the art. Normally the DNA encoding the heavy chain(s) is genetically engineered using the techniques described in Mutagenesis: a Practical Approach. Oligonucleotide-mediated mutagenesis is a preferred method for preparing substitution variants of the DNA encoding the two hybrid heavy chains. This technique is well known in the art as described by Adelman et al., (1983) DNA, 2:183.

2. Molecules with Variable Region Modifications

Each of the N-terminal VH and VL domains and C-terminal VH and VL domains of the molecule (e.g. antibody or antibody-like molecule) of the present invention comprises hypervariable regions CDR1, CDR2, and CDR3 sequences. In certain embodiments, one or more of these CDR sequences have conservative modifications of the amino acid sequences, and wherein the modified molecules retain or have enhanced binding properties as compared to the parent antibodies.

In addition, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al). The molecules (e.g. antibodies or antibody-like molecules) of the present invention can be modified by introducing such mutations to its variable region frameworks in order to improve the binding properties.

Another type of variable region modification is to mutate amino acid residues within the VH and/or VL CDR1, CDR2 and/or CDR3 domains to thereby improve one or more binding properties (e.g., affinity) of the molecule (e.g. antibody or antibody-like molecule) of interest, known as “affinity maturation.” Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Conservative modifications (as discussed above) can be introduced. The mutations may be amino acid substitutions, additions or deletions. Moreover, typically no more than one, two, three, four or five residues within a CDR region are altered.

Amino acid sequence variants of the present molecules can be prepared by introducing appropriate nucleotide changes into the encoding DNAs, or by synthesis of the desired variants. Such variants include, for example, deletions from, or insertions or substitutions of, residues within the amino acid sequences of present molecules. Any combination of deletion, insertion, and substitution is made to arrive at the final construct, provided that the final construct possesses the desired antigen-binding characteristics. The amino acid changes also may alter post-translational processes of the molecules, such as changing the number or position of glycosylation sites.

The present application includes variants of the molecules described herein and/or fragments thereof having amino acid conservative modifications in variable regions and/or constant regions.

3. Molecules with an Extended In Vivo Half-Life.

The present molecule can be further modified to have an extended half-life in vivo.

A variety of strategies can be used to extend the half life of the molecules of the present invention. For example, by chemical linkage to polyethyleneglycol (PEG), reCODE PEG, antibody scaffold, polysialic acid (PSA), hydroxyethyl starch (HES), albumin-binding ligands, and carbohydrate shields; by genetic fusion to proteins binding to serum proteins, such as albumin, IgG, FcRn, and transferring; by coupling (genetically or chemically) to other binding moieties that bind to serum proteins, such as nanobodies, Fabs, DARPins, avimers, affibodies, and anticalins; by genetic fusion to rPEG, albumin, domain of albumin, albumin-binding proteins, and Fc; or by incorporation into nancarriers, slow release formulations, or medical devices.

The molecules of the present invention having an increased half-life in vivo can also be generated introducing one or more amino acid modifications (i.e., substitutions, insertions or deletions) into an IgG constant domain, or FcRn binding fragment thereof (preferably a Fc or hinge Fc domain fragment). See, e.g., International Publication No. WO 98/23289; International Publication No. WO 97/34631; and U.S. Pat. No. 6,277,375.

Further, the molecules can be conjugated to albumin in order to make the molecules more stable in vivo or have a longer half life in vivo. The techniques are well-known in the art, see, e.g., International Publication Nos. WO 93/15199, WO 93/15200, and WO 01/77137; and European Patent No. EP 413,622.

The molecules of the present invention may also be fused to one or more human serum albumin (HSA) polypeptides, or a portion thereof. The use of albumin as a component of an albumin fusion protein as a carrier for various proteins has been suggested in WO 93/15199, WO 93/15200, and EP 413 622. The use of N-terminal fragments of HSA for fusions to polypeptides has also been proposed (EP 399 666). Accordingly, by genetically or chemically fusing or conjugating the molecules to albumin, can stabilize or extend the shelf-life, and/or to retain the molecule's activity for extended periods of time in solution, in vitro and/or in vivo. Additional methods pertaining to HSA fusions can be found, for example, in WO 2001077137 and WO 200306007, incorporated herein by reference. In a specific embodiment, the expression of the fusion protein is performed in mammalian cell lines, for example, CHO cell lines.

### 4. Antibody Conjugates

The present invention includes molecules (e.g. antibodies or antibody-like molecules) or the fragments thereof recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., (1991) Proc. Natl. Acad. Sci. USA 88:10535-10539; Zheng et al., (1995) J. Immunol. 154:5590-5600; and Vil et al., (1992) Proc. Natl. Acad. Sci. USA 89:11337-11341.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of molecules of the invention or fragments thereof (e.g., molecules or fragments thereof with higher affinities and lower dissociation rates). See, generally, U.S. Pat. Nos. 5,605,793, 5,811,238, 5,830,721, 5,834,252, and 5,837,458; Patten et al., (1997) Curr. Opinion Biotechnol. 8:724-33; Harayama, (1998) Trends Biotechnol. 16(2):76-82; Hansson et al., (1999) J. Mol. Biol. 287:265-76; and Lorenzo and Blasco, (1998) Biotechniques 24(2):308-313 (each of these patents and publications are hereby incorporated by reference in its entirety). The molecules described herein or fragments thereof may be altered by being subjected to random mutagenesis by error-prone PCR, random nucleotide insertion or other methods prior to recombination. A polynucleotide encoding a fragment of the present molecule may be recombined with one or more components, motifs, sections, parts, domains, fragments, etc. of one or more heterologous molecules.

Moreover, the present molecules or fragments thereof can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., (1989) Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., (1984) Cell 37:767), and the “flag” tag.

In other embodiments, the molecules of the present invention or fragments thereof are conjugated to a diagnostic or detectable agent. Such molecules can be useful for monitoring or prognosing the onset, development, progression and/or severity of a disease or disorder as part of a clinical testing procedure, such as determining the efficacy of a particular therapy. Such diagnosis and detection can accomplished by coupling the molecules to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (¹³¹O, ¹²⁵I, ¹²³I, and ¹²¹I,) carbon (¹⁴C), sulfur (³⁵S), tritium (³H), indium (115In, ¹¹³In, ¹¹²In, and ¹¹¹In,), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, 47Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, 169Yb, ⁵¹Cr, ⁵⁴Mn, ⁷⁵Se, ¹¹³Sn, and ¹¹⁷Tin; and positron emitting metals using various positron emission tomographies, and nonradioactive paramagnetic metal ions.

The present application further encompasses uses of the present molecules or fragments thereof conjugated to a therapeutic moiety. The molecules of the present invention or fragments thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells.

Further, the present molecule or fragment thereof may be conjugated to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, an anti-angiogenic agent; or, a biological response modifier such as, for example, a lymphokine.

In one embodiment, the present molecule, or a fragment thereof, is conjugated to a therapeutic moiety, such as a cytotoxin, a drug (e.g., an immunosuppressant) or a radiotoxin. Such conjugates are referred to herein as “immunoconjugates”. Immunoconjugates that include one or more cytotoxins are referred to as “immunotoxins.” A cytotoxin or cytotoxic agent includes any agent that is detrimental to (e.g., kills) cells. Examples include taxon, cytochalasin B, gramicidin D, ethidium bromide, emetine, mitomycin, etoposide, tenoposide, vincristine, vinblastine, t. colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), ablating agents (e.g., mechlorethamine, thioepa chloraxnbucil, meiphalan, carmustine (BSNU) and lomustine (CCNU), cyclothosphamide, busulfan, dibromomannitol, streptozotocin, mitomycin C, and cis-dichlorodiamine platinum (II) (DDP) cisplatin, anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). (See e.g., Seattle Genetics US20090304721).

Other examples of therapeutic cytotoxins that can be conjugated to the molecules of the present invention include duocarmycins, calicheamicins, maytansines and auristatins, and derivatives thereof. An example of a calicheamicin antibody conjugate is commercially available (Mylotarg™; Wyeth-Ayerst).

Cytoxins can be conjugated to the molecules of the invention using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

For further discussion of types of cytotoxins, linkers and methods for conjugating therapeutic agents to the molecules, see also Saito et al., (2003) Adv. Drug Deliv. Rev. 55:199-215; Trail et al., (2003) Cancer Immunol. Immunother. 52:328-337; Payne, (2003) Cancer Cell 3:207-212; Allen, (2002) Nat. Rev. Cancer 2:750-763; Pastan and Kreitman, (2002) Curr. Opin. Investig. Drugs 3:1089-1091; Senter and Springer, (2001) Adv. Drug Deliv. Rev. 53:247-264.

The molecules of the present invention also can be conjugated to a radioactive isotope to generate cytotoxic radiopharmaceuticals, also referred to as radioimmunoconjugates. Examples of radioactive isotopes that can be conjugated to molecules for use diagnostically or therapeutically include, but are not limited to, iodine¹³¹, indium¹¹¹, yttrium⁹⁰, and lutetium¹⁷⁷. Method for preparing radioimmunconjugates are established in the art. Examples of radioimmunoconjugates are commercially available, including Zevalin™ (DEC Pharmaceuticals) and Bexxar™ (Corixa Pharmaceuticals), and similar methods can be used to prepare radioimmunoconjugates using the molecules of the invention. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., (1998) Clin Cancer Res. 4(10):2483-90; Peterson et al., (1999) Bioconjug. Chem. 10(4):553-7; and Zimmerman et al., (1999) Nucl. Med. Biol. 26(8):943-50, each incorporated by reference in their entireties.

Techniques for conjugating therapeutic moieties to antibodies or antibody-like molecules are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., (1982) Immunol. Rev. 62:119-58.

The molecules may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### II. Methods of Making the Molecules of the Present Invention

To make the molecules of Format 1 or 2 of the invention, the C-terminus of the CH3 of the first antibody of the invention is functionally linked (e.g., by chemical coupling, genetic fusion, non-covalent association or otherwise) to VH or VL or a tandem thereof of the second antibody. In one embodiment, the C-terminus of the CH3 of a first patent antibody of the invention is functionally linked to the N-terminus of VH or VL or a tandem thereof of the second antibody. Additionally, to make the molecule of Format 3, the C-terminus of CH3 and the N-terminus of CH2 is functional linked to VH or VL domains of the first and second antibodies.

These functional linkages can be accomplished using methods known in the art. A variety of coupling or cross-linking agents can be used for covalent conjugation. Examples of cross-linking agents include protein A, carbodiimide, N-succinimidyl-5-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohexane-1-carboxylate (sulfo-SMCC) (see e.g., Karpovsky et al., (1984) J. Exp. Med. 160:1686; Liu et al. (1985) Proc. Natl. Acad. Sci. USA 82:8648). Other methods include those described in Paulus (1985) Behring Ins. Mitt. No. 78:118-132; Brennan et al., (1985) Science 229:81-83), and Glennie et al., (1987) J. Immunol. 139: 2367-2375). Conjugating agents are SATA and sulfo-SMCC, both available from Pierce Chemical Co. (Rockford, Ill.).

Alternatively, the present molecules can be generated recombinantly by introducing DNA constructs encoding the desired molecules into expression vectors and expressing and assembling the desired molecules in the same host cells.

### A. Preparing Parent Antibodies

The first step of producing the present molecules is preparing the parent antibodies (the first and the second antibodies described herein). If the molecules are produced recombinantly, the nucleic acid molecules encoding the first and second (parent) antibodies need to be prepared first.

A variety of antibodies can be used as the parent antibodies for the present molecules, including monoclonal antibodies, chimeric antibodies, humanized antibodies, human antibodies, multi-specific antibodies and antibody conjugates. The parent antibodies can be of any isotype (e.g., IgG, IgE, IgM, IgD, IgA and IgY), class (e.g., IgG1, IgG2, IgG3, IgG4, IgA1 and IgA2) or subclass. These antibodies recognize a variety of polypeptides known in the art, preferably the polypeptides that play important roles in biological functions of the mammalian cells.

Monoclonal antibodies (mAbs) can be produced by a variety of techniques, including conventional monoclonal antibody methodology e.g., the standard somatic cell hybridization technique of Kohler and Milstein, (1975) Nature 256: 495. Many techniques for producing monoclonal antibody can be employed e.g., viral or oncogenic transformation of B lymphocytes.

An animal system for preparing hybridomas is the murine system. Hybridoma production in the mouse is a well established procedure. Immunization protocols and techniques for isolation of immunized splenocytes for fusion are known in the art. Fusion partners (e.g., murine myeloma cells) and fusion procedures are also known.

Chimeric or humanized antibodies used in the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

In a certain embodiment, the parent antibodies of the invention are human monoclonal antibodies. Such human monoclonal antibodies can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb Mouse® (Medarex, Inc.) contains human immunoglobulin gene miniloci that encode un-rearranged human heavy (μ and γ) and κ light chain immunoglobulin sequences, together with targeted mutations that inactivate the endogenous μ and κ chain loci (see e.g., Lonberg, et al., (1994) Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg et al., (1994) supra; reviewed in Lonberg, (1994) Handbook of Experimental Pharmacology 113:49-101; Lonberg and Huszar, (1995) Intern. Rev. Immunol. 13: 65-93, and Harding and Lonberg, (1995) Ann. N.Y. Acad. Sci. 764:536-546). The preparation and use of HuMAb mice, and the genomic modifications carried by such mice, is further described in Taylor et al., (1992) Nucleic Acids Research 20:6287-6295; Chen et al., (1993) International Immunology 5: 647-656; Tuaillon et al., (1993) Proc. Natl. Acad. Sci. USA 94:3720-3724; Choi et al., (1993) Nature Genetics 4:117-123; Chen et al., (1993) EMBO J. 12:821-830; Tuaillon et al., (1994) J. Immunol. 152:2912-2920; Taylor et al., (1994) International Immunology 579-591; and Fishwild et al., (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92103918, WO 93/12227, WO 94/25585, WO 97113852, WO 98/24884 and WO 99/45962, all to Lonberg and Kay; and PCT Publication No. WO 01/14424 to Korman et al.

In another embodiment, human antibodies used in the present invention can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in PCT Publication WO 02/43478 to Ishida et al.

Still further, alternative transgenic animal systems expressing human immunoglobulin genes are available in the art and can be used to raise human antibodies used in the present invention. For example, an alternative transgenic system referred to as the Xenomouse (Abgenix, Inc.) can be used. Such mice are described in, e.g., U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise the human antibodies used in the invention. For example, mice carrying both a human heavy chain transchromosome and a human light chain tranchromosome, referred to as “TC mice” can be used; such mice are described in Tomizuka et al., (2000) Proc. Natl. Acad. Sci. USA 97:722-727. Furthermore, cows carrying human heavy and light chain transchromosomes have been described in the art (Kuroiwa et al., (2002) Nature Biotechnology 20:889-894) and can be used to raise human antibodies used in the present application.

Human monoclonal antibodies can also be prepared using phage display methods for screening libraries of human immunoglobulin genes. Such phage display methods for isolating human antibodies are established in the art or described in the examples below. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies used in the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

The parent antibody of the present invention can also be a bi-specific antibody comprising two heavy chains, each of which is capable of binding two different epitopes or antigens. Method of making bispecific antibodies are known in the art and discussed in the present application.

### B. Methods of Producing Molecules of the Present Invention Recombinantly

In one embodiment, the present application provides a method of producing the present molecule of Format 1 recombinantly, comprising: 1) producing a DNA construct comprising a nucleic acid molecule encoding a heavy chain comprising a heavy chain of a first antibody connected at its C-terminus a) a VL, b) a tandem of VLs, c) a VH or d) a tandem of VHs of a second antibody; 2) introducing said DNA construct into an expression vector; 3) co-transfecting said expression vector in a host cell together with an expression vector comprising a nucleic acid molecule encoding the light chain of said first antibody; and 4) expressing and assemble the molecule in a host cell.

The molecule of the present invention is produced by first combining a DNA sequence encoding a single heavy chain of the first antibody with a DNA sequence encoding the heavy chain variable region or the light chain variable region or a tandem thereof of the second antibody as described herein. Normally these sequences are combined such that the C-terminus of the constant region of the single heavy chain is linked to the N-terminus of the VH or VL or a tandem thereof of the second antibody through a short flexible linker region so that a hybrid single heavy chain is created and will become the single heavy chain of the molecule of the present invention. In an exemplary embodiment, two DNA sequences are produced at this stage. One DNA sequence encodes a single heavy chain of the first antibody connected with a VH or a tandem of VH of the second antibody, while the other DNA sequence encodes a single heavy chain of the first antibody connected with a VL or a tandem of VL of the second antibody. The created DNA sequences are then separately put into an expression vector.

The similar approach can be used to produce the molecule of “one-arm” format (Format 2). Two DNA sequences are produced at this stage. One DNA sequence encodes a single heavy chain of the first antibody connected with a VH (or VL) or a tandem of VH (or VL) of the second antibody, while the other DNA sequence encodes a partial heavy chain of the first antibody connected with a VL (or VH) or a tandem of VL (or VH) of the second antibody. The partial heavy chain consists essentially of hinge region, CH2 and CH3 domains. The created two DNA sequences are then put into separate expression vectors.

For the molecules of Format 1 and/or Format 2, the DNA sequence encoding the light chain of the first antibody is also produced and put into a separate expression vector. The expression vectors are then co-transfected into a host cell at a ratio giving rise to optimal assembly. The encoded heavy chains and light chains are expressed in the host cell and assemble into functional molecules.

Similar methods can also be used to produce molecules of Format 3. In particular, the method comprises: 1) producing a DNA construct comprising a nucleic acid molecule encoding a single chain of the molecule of Format 3, which comprises a fragment of CH2-CH3 having the CH2 domain connected at its N-terminus to a VH (or VL) of a first antibody and having the CH3 domain connected at its C-terminus to a VH (or VL) of a second antibody 2) introducing said DNA construct into an expression vector; 3) transfecting said expression vector into a host cell and 4) expressing and assemble the molecule in a host cell.

Desired mutations on the variable region or the constant region of the molecule described herein, such as, for enhancing hetero-dimerization, can be introduced at this stage as described herein.

The DNA sequences can be produced by de novo solid-phase DNA synthesis or by PCR mutagenesis of an existing sequence (e.g., sequences as described in the Examples below) encoding heavy or light chains of the present molecules. Direct chemical synthesis of nucleic acids can be accomplished by methods known in the art, such as the phosphotriester method of Narang et al., (1979) Meth. Enzymol. 68:90; the phosphodiester method of Brown et al., (1979) Meth. Enzymol. 68:109; the diethylphosphoramidite method of Beaucage et al., (1981) Tetra. Lett., 22:1859; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif., 1990; Mattila et al., (1991) Nucleic Acids Res. 19:967; and Eckert et al., (1991) PCR Methods and Applications 1:17.

Also provided in the invention are expression vectors and host cells for producing the molecules described above. Various expression vectors can be employed to express the polynucleotides encoding chains or binding fragments of the molecule. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., (1997) Nat Genet 15:345). For example, nonviral vectors useful for expression of the polynucleotides and polypeptides in mammalian (e.g., human) cells include pThioHis A, B & C, pcDNA3.1/His, pEBVHis A, B & C, (Invitrogen, San Diego, Calif.), MPSV vectors, and numerous other vectors known in the art for expressing other proteins. Useful viral vectors include vectors based on retroviruses, adenoviruses, adeno associated viruses, herpes viruses, vectors based on SV40, papilloma virus, HBP Epstein Barr virus, vaccinia virus vectors and Semliki Forest virus (SFV). See, Brent et al., (1995) supra; Smith, Annu. Rev. Microbiol. 49:807; and Rosenfeld et al., (1992) Cell 68:143.

The choice of expression vector depends on the intended host cells in which the vector is to be expressed. Typically, the expression vectors contain a promoter and other regulatory sequences (e.g., enhancers) that are operably linked to the polynucleotides encoding an antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of the heavy chains and light chains of the multi-specific molecules. These elements typically include an ATG initiation codon and adjacent ribosome binding site or other sequences. In addition, the efficiency of expression may be enhanced by the inclusion of enhancers appropriate to the cell system in use (see, e.g., Scharf et al., (1994) Results Probl. Cell Differ. 20:125; and Bittner et al., (1987) Meth. Enzymol., 153:516). For example, the SV40 enhancer or CMV enhancer may be used to increase expression in mammalian host cells.

The expression vectors may also provide a secretion signal sequence position to form a fusion protein with polypeptides encoded by inserting the above-described sequences of heavy chain and/or light chain or fragments thereof. More often, the inserted antibody or antibody-like molecule sequences are linked to a signal sequences before inclusion in the vector. Vectors to be used to receive sequences encoding light and heavy chain variable domains sometimes also encode constant regions or parts thereof. Such vectors allow expression of the variable regions as fusion proteins with the constant regions thereby leading to production of intact antibodies or antibody-like molecules or fragments thereof. Typically, such constant regions are human.

The host cells for harboring and expressing the present molecules can be either prokaryotic or eukaryotic. E. coli is one prokaryotic host useful for cloning and expressing the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express the antibody of the invention. Insect cells in combination with baculovirus vectors can also be used.

In some preferred embodiments, mammalian host cells are used to express and produce the molecules of the present invention. For example, they can be either a hybridoma cell line expressing endogenous immunoglobulin genes (e.g., the 1D6.C9 myeloma hybridoma clone as described in the Examples) or a mammalian cell line harboring an exogenous expression vector (e.g., the SP2/0 myeloma cells exemplified below). These include any normal mortal or normal or abnormal immortal animal or human cell. For example, a number of suitable host cell lines capable of secreting intact immunoglobulins have been developed including the CHO cell lines, various Cos cell lines, HeLa cells, myeloma cell lines, transformed B-cells and hybridomas. The use of mammalian tissue cell culture to express polypeptides is discussed generally in, e.g., Winnacker, FROM GENES TO CLONES, VCH Publishers, N.Y., N.Y., 1987. Expression vectors for mammalian host cells can include expression control sequences, such as an origin of replication, a promoter, and an enhancer (see, e.g., Queen et al., (1986) Immunol. Rev. 89:49-68), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts. (See generally Sambrook, et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, (1997) Cell 88:223), agent-enhanced uptake of DNA, and ex vivo transduction. For long-term, high-yield production of recombinant proteins, stable expression will often be desired. For example, cell lines which stably express antibody chains or binding fragments can be prepared using expression vectors of the invention which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

The present molecule preferably is generally recovered from the culture medium as a secreted polypeptide, although it also may be recovered from host cell lysate when directly produced without a secretory signal. If the molecule is membrane-bound, it can be released from the membrane using a suitable detergent solution (e.g. Triton-X 100).

When the molecule is produced in a recombinant cell other than one of human origin, it is completely free of proteins or polypeptides of human origin. However, it is necessary to purify the molecule from recombinant cell proteins or polypeptides to obtain preparations that are substantially homogeneous as to heteromultimer. As a first step, the culture medium or lysate is normally centrifuged to remove particulate cell debris. The produced molecules can be conveniently purified by hydroxylapatite chromatography, gel electrophoresis, dialysis, or affinity chromatography, with affinity chromatography being the preferred purification technique. Other techniques for protein purification such as fractionation on an ion-exchange column, ethanol precipitation, reverse phase HPLC, chromatography on silica, chromatography on heparin Sepharose, chromatography on an anion or cation exchange resin (such as a polyaspartic acid column), chromatofocusing, SDS-PAGE, and ammonium sulfate precipitation are also available.

### III. Use of the Molecules of the Present Invention

### A. Diagnostic and Therapeutic Use

Depending on the antigens that recognized by the molecules of the present invention, the present molecules have many diagnostic and therapeutic applications. For instance, they can be used for enzyme immunoassay, with N-terminal arms binding a specific epitope on the enzyme and C-terminal arms binding the immobilizing matrix. The enzyme immunoassay using antibody-like molecules is discussed by Nolan et al. (Nolan et al., (1990) Biochem. Biophys. Acta. 1040:1-11). molecules can also be used for diagnosis of various diseases such as cancer (Songsivilai et al., (1990) Clin. Exp. Immunol. 79:315). In particular, one binding site of the molecule can bind a tumor associated antigen and the other binding site can bind a detectable marker described herein, for example, a chelator which tightly binds a radionuclide. (Le Doussal et al., (1992) Int. J. Cancer Suppl. 7:58-62 and Le Doussal et al., (1993) J. Nucl. Med. 34:1662-1671; Stickney et al., (1995) Cancer Res. 51:6650-6655).

The present molecules find therapeutic uses for treating various human diseases, for example, cancer, autoimmune diseases, and infectious diseases, etc.

For instance, with N-terminal arms binding a tumor target or a pathogen target and C-terminal arms binding a T cell receptor or Fc receptor, the present molecules are capable of killing tumor cells or pathogens by using the patient's immune defense system using the approach discussed in Segal et al., Chem. Immunol. 47:179 (1989) and Segal et al., Biologic Therapy of Cancer 2(4) DeVita et al. eds. J. B. Lippincott, Philadelphia (1992) p. 1.

Similarly, the present molecules can also mediate killing by T cells, for example by linking the CD3 complex on T cells to a tumor-associated antigen.

The present molecules may also be used as fibrinolytic agents or vaccine adjuvants. Furthermore, the antibodies or antibody-like molecules may be used in the treatment of infectious diseases (e.g. for targeting of effector cells to virally infected cells such as HIV or influenza virus or protozoa such as Toxoplasma gondii), used to deliver immunotoxins to tumor cells, or target immune complexes to cell surface receptors (Romet-Lemonne, Fanger and Segal Eds., Lienhart (1991) p. 249.). The present molecules may also be used to deliver immunotoxin to tumor cells.

### B. Pharmaceutical Compositions

To prepare pharmaceutical or sterile compositions including the molecule of the present invention, the molecule is mixed with a pharmaceutically acceptable carrier or excipient.

Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: eral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Selecting an administration regimen for a therapeutic depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom, et al. (1999) New Engl. J. Med. 341:1966-1973; Slamon, et al. (2001) New Engl. J. Med. 344:783-792; Beniaminovitz, et al. (2000) New Engl. J. Med. 342:613-619; Ghosh, et al. (2003) New Engl. J. Med. 348:24-32; Lipsky, et al. (2000) New Engl. J. Med. 343:1594-1602).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors known in the medical arts.

Compositions comprising the molecules or fragments thereof of the present application can be provided by continuous infusion, or by doses at intervals of, e.g., one day, one week, or 1-7 times per week. Doses may be provided intravenously, subcutaneously, topically, orally, nasally, rectally, intramuscular, intracerebrally, or by inhalation. A specific dose protocol is one involving the maximal dose or dose frequency that avoids significant undesirable side effects. A total weekly dose may be at least 0.05 μg/kg body weight, at least 0.2 μg/kg, at least 0.5 μg/kg, at least 1 μg/kg, at least 10 μg/kg, at least 100 μg/kg, at least 0.2 mg/kg, at least 1.0 mg/kg, at least 2.0 mg/kg, at least 10 mg/kg, at least 25 mg/kg, or at least 50 mg/kg (see, e.g., Yang, et al. (2003) New Engl. J. Med. 349:427-434; Herold, et al. (2002) New Engl. J. Med. 346:1692-1698; Liu, et al. (1999) J. Neurol. Neurosurg. Psych. 67:451-456; Portielji, et al. (2003) Cancer Immunol. Immunother. 52:133-144). The desired dose of the molecules or fragments thereof is about the same as for an antibody or polypeptide, on a moles/kg body weight basis. The desired plasma concentration of the molecules or fragments thereof is about, on a moles/kg body weight basis. The dose may be at least 15 μg at least 20 μg, at least 25 μg, at least 30 μg, at least 35 μg, at least 40 μg, at least 45 μg, at least 50 μg, at least 55 μg, at least 60 μg, at least 65 μg, at least 70 μg, at least 75 μg, at least 80 μg, at least 85 μg, at least 90 μg, at least 95 μg, or at least 100 μg. The doses administered to a subject may number at least 1, 2, 3, 4, 5, 6, 7, 8, 9, 10, 11, or 12, or more.

For the molecules or fragments thereof of the invention, the dosage administered to a patient may be 0.0001 mg/kg to 100 mg/kg of the patient's body weight. The dosage may be between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight.

The dosage of the molecules or fragments thereof of the present application may be calculated using the patient's weight in kilograms (kg) multiplied by the dose to be administered in mg/kg. The dosage of the molecules or fragments thereof, of the present application may be 150 μg/kg or less, 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight.

Unit dose of the molecules or fragments thereof of the present application may be 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 m g, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

The dosage of the molecules or fragments thereof of the present application may achieve a serum titer of at least 0.1 μg/ml, at least 0.5 μg/ml, at least 1 μg/ml, at least 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml in a subject. Alternatively, the dosage of the molecule or fragments thereof, of the present application may achieve a serum titer of at least 0.1 μg/ml, at least 0.5 μg/ml, at least 1 μg/ml, at least, 2 μg/ml, at least 5 μg/ml, at least 6 μg/ml, at least 10 μg/ml, at least 15 μg/ml, at least 20 μg/ml, at least 25 μg/ml, at least 50 μg/ml, at least 100 μg/ml, at least 125 μg/ml, at least 150 μg/ml, at least 175 μg/ml, at least 200 μg/ml, at least 225 μg/ml, at least 250 μg/ml, at least 275 μg/ml, at least 300 μg/ml, at least 325 μg/ml, at least 350 μg/ml, at least 375 μg/ml, or at least 400 μg/ml in the subject.

Doses of the molecules or fragments thereof of the application may be repeated and the administrations may be separated by at least 1 day, 2 days, 3 days, 5 days, 10 days, 15 days, 30 days, 45 days, 2 months, 75 days, 3 months, or at least 6 months.

An effective amount for a particular patient may vary depending on factors such as the condition being treated, the overall health of the patient, the method route and dose of administration and the severity of side effects (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

The route of administration may be by, e.g., topical or cutaneous application, injection or infusion by intravenous, intraperitoneal, intracerebral, intramuscular, intraocular, intraarterial, intracerebrospinal, intralesional, or by sustained release systems or an implant (see, e.g., Sidman et al. (1983) Biopolymers 22:547-556; Langer, et al. (1981) J. Biomed. Mater. Res. 15:167-277; Langer (1982) Chem. Tech. 12:98-105; Epstein, et al. (1985) Proc. Natl. Acad. Sci. USA 82:3688-3692; Hwang, et al. (1980) Proc. Natl. Acad. Sci. USA 77:4030-4034; U.S. Pat. Nos. 6,350,466 and 6,316,024). Where necessary, the composition may also include a solubilizing agent and a local anesthetic such as lidocaine to ease pain at the site of the injection. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

A composition of the present invention may also be administered via one or more routes of administration using one or more of a variety of methods known in the art. As will be appreciated by the skilled artisan, the route and/or mode of administration will vary depending upon the desired results. Selected routes of administration for molecules or fragments thereof of the invention include intravenous, intramuscular, intradermal, intraperitoneal, subcutaneous, spinal or other eral routes of administration, for example by injection or infusion. eral administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the present application can be administered via a non-eral route, such as a topical, epidermal or mucosal route of administration, for example, intranasally, orally, vaginally, rectally, sublingually or topically. In one embodiment, the molecules or fragments thereof of the invention is administered by infusion. In another embodiment, the multispecific epitope binding protein of the invention is administered subcutaneously.

If the molecules or fragments thereof of the invention are administered in a controlled release or sustained release system, a pump may be used to achieve controlled or sustained release (see Langer, supra; Sefton, 1987, CRC Crit. Ref Biomed. Eng. 14:20; Buchwald et al., 1980, Surgery 88:507; Saudek et al., 1989, N. Engl. J. Med. 321:574). Polymeric materials can be used to achieve controlled or sustained release of the therapies of the invention (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more molecules or fragments thereof of the present application. See, e.g., U.S. Pat. No. 4,526,938, PCT publication WO 91/05548, PCT publication WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

If the molecules or fragments thereof of the invention are administered topically, they can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in some instances, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the compositions comprising the molecules or fragments thereof are administered intranasally, it can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

Methods for co-administration or treatment with a second therapeutic agent, e.g., a cytokine, steroid, chemotherapeutic agent, antibiotic, or radiation, are known in the art (see, e.g., Hardman, et al. (eds.) (2001) Goodman and Gilman's The Pharmacological Basis of Therapeutics, 10.sup.th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of therapeutic may decrease the symptoms by at least 10%; by at least 20%; at least about 30%; at least 40%, or at least 50%.

Additional therapies (e.g., prophylactic or therapeutic agents), which can be administered in combination with the molecules or fragments thereof of the present application may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the molecules or fragments thereof of the invention. The two or more therapies may be administered within one same patient visit.

The molecules or fragments thereof of the invention and the other therapies may be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

In certain embodiments, the molecules or fragments thereof of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p 120 (Schreier et al (1994) J. Biol. Chem. 269:9090); see also K. Keinanen; M. L. Laukkanen (1994) FEBS Lett. 346:123; J. J. Killion; I. J. Fidler (1994) Immunomethods 4:273.

The present application provides protocols for the administration of pharmaceutical composition comprising molecules or fragments thereof of the present application alone or in combination with other therapies to a subject in need thereof. The therapies (e.g., prophylactic or therapeutic agents) of the combination therapies of the present invention can be administered concomitantly or sequentially to a subject. The therapy (e.g., prophylactic or therapeutic agents) of the combination therapies of the present invention can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

The therapies (e.g., prophylactic or therapeutic agents) of the combination therapies of the invention can be administered to a subject concurrently. The term “concurrently” is not limited to the administration of therapies (e.g., prophylactic or therapeutic agents) at exactly the same time, but rather it is meant that a pharmaceutical composition comprising molecules or fragments thereof of the present application are administered to a subject in a sequence and within a time interval such that the molecules of the invention can act together with the other therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route. In various embodiments, the therapies (e.g., prophylactic or therapeutic agents) are administered to a subject less than 15 minutes, less than 30 minutes, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In other embodiments, two or more therapies (e.g., prophylactic or therapeutic agents) are administered to a within the same patient visit.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

IV. Molecules Having Binding Specificity for HER1 and/or HER2

The HER family of receptor tyrosine kinases are important mediators of cell growth, differentiation and survival. The receptor family includes four distinct members including epidermal growth factor receptor (EGFR, ErbB1, or HER1), HER2 (ErbB2 or p185.sup.neu), HER3 (ErbB3) and HER4 (ErbB4 or tyro2).

EGFR, encoded by the erbB 1 gene, has been causally implicated in human malignancy. In particular, increased expression of EGFR has been observed in breast, bladder, lung, head, neck and stomach cancer as well as glioblastomas. Increased EGFR receptor expression is often associated with increased production of the EGFR ligand, transforming growth factor alpha (TGF-.alpha.), by the same tumor cells resulting in receptor activation by an autocrine stimulatory pathway. Baselga and Mendelsohn, (1994) Pharmac. Ther. 64:127-154. Monoclonal antibodies directed against the EGFR or its ligands, TGF-α and EGF, have been evaluated as therapeutic agents in the treatment of such malignancies. See, e.g., Masui et al. (1984) Cancer Research 44:1002-1007; and Wu et al. (1995) J. Clin. Invest. 95:1897-1905.

The second member of the HER family, p185.^{neu}, was originally identified as the product of the transforming gene from neuroblastomas of chemically treated rats. The activated form of the neu proto-oncogene results from a point mutation (valine to glutamic acid) in the transmembrane region of the encoded protein. Amplification of the human homolog of neu is observed in breast and ovarian cancers and correlates with a poor prognosis (Slamon et al., (1987) Science, 235:177-182; Slamon et al., (1989) Science, 244:707-712; and U.S. Pat. No. 4,968,603). To date, no point mutation analogous to that in the neu proto-oncogene has been reported for human tumors. Overexpression of HER2 (frequently but not uniformly due to gene amplification) has also been observed in other carcinomas including carcinomas of the stomach, endometrium, salivary gland, lung, kidney, colon, thyroid, pancreas and bladder. See, among others, King et al., (1985) Science, 229:974; Yokota et al., (1986) Lancet: 1:765-767; Fukushige et al., (1986) Mol Cell Biol., 6:955-958; Guerin et al., (1988) Oncogene Res., 3:21-31; Cohen et al., (1989) Oncogene, 4:81-88; Yonemura et al., (1991) Cancer Res., 51:1034. A recombinant humanized version of the murine HER2 antibody 4D5 (huMAb4D5-8, rhuMAb HER2, trastuzumab or HERCEPTIN®; U.S. Pat. No. 5,821,337) is clinically active in patients with HER2-overexpressing metastatic breast cancers that have received extensive prior anti-cancer therapy (Baselga et al., (1996) J. Clin. Oncol. 14:737-744). Trastuzumab received marketing approval from the Food and Drug Administration Sep. 25, 1998 for the treatment of patients with metastatic breast cancer whose tumors overexpress the HER2 protein. In addition, Pertuzumab (also known as recombinant human monoclonal antibody 2C4; OMNITARG®) is another clinically approved anti-HER2 antibody and represents the first in a new class of agents known as HER dimerization inhibitors (HDI) and functions to inhibit the ability of HER2 to form active heterodimers with other HER receptors (such as EGFR/HER1, HER3 and HER4) and is active irrespective of HER2 expression levels. See, for example, Harari and Yarden, (2000) Oncogene 19:6102-14; Yarden and Sliwkowski, (2001) Nat. Rev. Mol. Cell. Biol. 2:127-37; Sliwkowski, (2003) Nat. Struct. Biol. 10:158-9; Cho et al., (2003) Nature 421:756-60; and Malik et al., (2003) Pro Am Soc Cancer Res 44:176-7.

Other HER2 antibodies with various properties have been described in Tagliabue et al., (1991) Int. J. Cancer 47:933-937; McKenzie et al., (1989) Oncogene 4:543-548; Maier et al., (1991) Cancer Res. 51:5361-5369; Bacus et al., (1990) Molecular Carcinogenesis 3:350-362; Stancovski et al., (1991) PNAS (USA) 88:8691-8695; Bacus et al., (1992) Cancer Research 52:2580-2589; Xu et al., (1993) Int. J. Cancer 53:401-408; WO94/00136; Kasprzyk et al., (1992) Cancer Research 52:2771-2776; Hancock et al., (1991) Cancer Res. 51:4575-4580; Shawver et al., (1994) Cancer Res. 54:1367-1373; Arteaga et al., (1994) Cancer Res. 54:3758-3765; Harwerth et al., (1992) J. Biol. Chem. 267:15160-15167; U.S. Pat. No. 5,783,186; and Klapper et al., (1997) Oncogene 14:2099-2109. Rst et al., (1990) Gynecol. Oncol., 38:364; Weiner et al., (1990) Cancer Res., 50:421-425; Kern et al., (1990) Cancer Res., 50:5184; Park et al., (1989) Cancer Res., 49:6605; Zhau et al., (1990) Mol. Carcinog., 3:254-257; Aasland et al., (1988) Br. J. Cancer 57:358-363; Williams et al., (1991) Pathobiology 59:46-52; and McCann et al., (1990) Cancer, 65:88-92. HER2 may be overexpressed in prostate cancer (Gu et al., (1996) Cancer Lett. 99:185-9; Ross et al., (1997) Hum. Pathol. 28:827-33; Ross et al., (1997) Cancer 79:2162-70; and Sadasivan et al., (1993) J. Urol. 150:126-31).

Patent publications related to HER antibodies include: U.S. Pat. No. 5,677,171, U.S. Pat. No. 5,720,937, U.S. Pat. No. 5,720,954, U.S. Pat. No. 5,725,856, U.S. Pat. No. 5,770,195, U.S. Pat. No. 5,772,997, U.S. Pat. No. 6,165,464, U.S. Pat. No. 6,387,371, U.S. Pat. No. 6,399,063, US2002/0192211A1, U.S. Pat. No. 6,015,567, U.S. Pat. No. 6,333,169, U.S. Pat. No. 4,968,603, U.S. Pat. No. 5,821,337, U.S. Pat. No. 6,054,297, U.S. Pat. No. 6,407,213, U.S. Pat. No. 6,719,971, U.S. Pat. No. 6,800,738, US2004/0236078A1, U.S. Pat. No. 5,648,237, U.S. Pat. No. 6,267,958, U.S. Pat. No. 6,685,940, U.S. Pat. No. 6,821,515, WO98/17797, U.S. Pat. No. 6,127,526, U.S. Pat. No. 6,333,398, U.S. Pat. No. 6,797,814, U.S. Pat. No. 6,339,142, U.S. Pat. No. 6,417,335, U.S. Pat. No. 6,489,447, WO99/31140, US2003/0147884A1, US2003/0170234A1, US2005/0002928A1, U.S. Pat. No. 6,573,043, US2003/0152987A1, WO99/48527, US2002/0141993A1, WO01/00245, US2003/0086924, US2004/0013667A1, WO00/69460, WO01/00238, WO01/15730, U.S. Pat. No. 6,627,196B 1, U.S. Pat. No. 6,632,979B 1, WO01/00244, US2002/0090662A1, WO01/89566, US2002/0064785, US2003/0134344, WO 04/24866, US2004/0082047, US2003/0175845A1, WO03/087131, US2003/0228663, WO2004/008099A2, US2004/0106161, WO2004/048525, US2004/0258685A1, U.S. Pat. No. 5,985,553, U.S. Pat. No. 5,747,261, U.S. Pat. No. 4,935,341, U.S. Pat. No. 5,401,638, U.S. Pat. No. 5,604,107, WO 87/07646, WO 89/10412, WO 91/05264, EP 412,116 B1, EP 494,135 B1, U.S. Pat. No. 5,824,311, EP 444,181 B 1, EP 1,006,194 A2, US 2002/0155527A1, WO 91/02062, U.S. Pat. No. 5,571,894, U.S. Pat. No. 5,939,531, EP 502,812 B1, WO 93/03741, EP 554,441 B1, EP 656,367 A1, U.S. Pat. No. 5,288,477, U.S. Pat. No. 5,514,554, U.S. Pat. No. 5,587,458, WO 93/12220, WO 93/16185, U.S. Pat. No. 5,877,305, WO 93/21319, WO 93/21232, U.S. Pat. No. 5,856,089, WO 94/22478, U.S. Pat. No. 5,910,486, U.S. Pat. No. 6,028,059, WO 96/07321, U.S. Pat. No. 5,804,396, U.S. Pat. No. 5,846,749, EP 711,565, WO 96/16673, U.S. Pat. No. 5,783,404, U.S. Pat. No. 5,977,322, U.S. Pat. No. 6,512,097, WO 97/00271, U.S. Pat. No. 6,270,765, U.S. Pat. No. 6,395,272, U.S. Pat. No. 5,837,243, WO 96/40789, U.S. Pat. No. 5,783,186, U.S. Pat. No. 6,458,356, WO 97/20858, WO 97/38731, U.S. Pat. No. 6,214,388, U.S. Pat. No. 5,925,519, WO 98/02463, U.S. Pat. No. 5,922,845, WO 98/18489, WO 98/33914, U.S. Pat. No. 5,994,071, WO 98/45479, U.S. Pat. No. 6,358,682 B1, US 2003/0059790, WO 99/55367, WO 01/20033, US 2002/0076695 A1, WO 00/78347, WO 01/09187, WO 01/21192, WO 01/32155, WO 01/53354, WO 01/56604, WO 01/76630, WO02/05791, WO 02/11677, U.S. Pat. No. 6,582,919, US2002/0192652A1, US 2003/0211530 A1, WO 02/44413, US 2002/0142328, U.S. Pat. No. 6,602,670 B2, WO 02/45653, WO 02/055106, US 2003/0152572, US 2003/0165840, WO 02/087619, WO 03/006509, WO03/012072, WO 03/028638, US 2003/0068318, WO 03/041736, EP 1,357,132, US 2003/0202973, US 2004/0138160, U.S. Pat. No. 5,705,157, U.S. Pat. No. 6,123,939, EP 616,812 B1, US 2003/0103973, US 2003/0108545, U.S. Pat. No. 6,403,630 B1, WO 00/61145, WO 00/61185, U.S. Pat. No. 6,333,348 B1, WO 01/05425, WO 01/64246, US 2003/0022918, US 2002/0051785 A1, U.S. Pat. No. 6,767,541, WO 01/76586, US 2003/0144252, WO 01/87336, US 2002/0031515 A1, WO 01/87334, WO 02/05791, WO 02/09754, US 2003/0157097, US 2002/0076408, WO 02/055106, WO 02/070008, WO 02/089842 and WO 03/86467.

The anti-HER1 and anti-HER2 antibodies, including the exemplary antibodies described in the above references, can be used as parent antibodies (first or second antibody) to produce molecules of the present invention, for example, the molecules of Format 1, 2, 3, or 4. A variety of HER antibodies can be used as the parent antibodies for the present molecules, including monoclonal antibodies, chimeric antibodies, humanized antibodies, human antibodies, multi-specific antibodies and antibody conjugates. The antibodies can be of any isotype (e.g., IgG, IgE, IgM, IgD, IgA and IgY), class (e.g., IgG1, IgG2, IgG3, IgG4, IgA1 and IgA2) or subclass.

Thus, in an exemplary embodiment, the present invention provides a molecule of the present formats (Format 1, 2 or 3) having a dual binding or multiple binding specificities with HER1 and HER2.

In one embodiment, an anti-HER1 and an anti-HER2 antibody are used as the parent antibodies to generate a molecule of Format 1. In one aspect, the heavy chain of an anti-HER1 antibody is connected at its C-terminus with a VH or VL, or a tandem thereof of an anti-HER2 antibody. In a preferred embodiment, one heavy chain of an anti-HER1 antibody is connected with a VH or a tandem thereof of an anti-HER2 antibody, while the other heavy chain of the anti-HER1 antibody is connected with VL or a tandem of VL of the anti-HER2 antibody. Accordingly, a HER1/HER2 molecule is so created having two N-terminal binding arms specifically for HER1 and one C-terminal arm specifically binding HER2. In another aspect, the present invention also includes a HER2/HER1 molecule of Format 1 having two N-terminal binding arms specifically for HER2 and one C-terminal binding arm specifically binding HER1. This molecule can be produced by using an anti-HER2 antibody as the first antibody and an anti-HER1 antibody as the second antibody.

In yet another aspect, the present invention includes a molecule that binds to at least two, or three, different epitopes on HER1 and/or HER2. In one exemplary embodiment, the molecule binds two identical epitopes of HER1 (or HER2) at N-terminal arms and a different epitope of HER1 (or HER2) in the C-terminal arms. In another exemplary emboiment, the molecule binds two different epitopes of HER1 (or HER2) at the N-terminal arms and a third different epitope of HER1 (or HER2) at its C-terminal arms. In yet another exemplary embodiment, the molecule binds two identical epitopes of HER1 (or HER2) at the N-terminal arms and HER2 (or HER1) at its C-terminal arms. In yet another exemplary embodiment, the molecule binds two different epitopes of HER1 (or HER2) at the N-terminal arms and HER2 (or HER1) at its C-terminal arms.

In another embodiment, an anti-HER1 and/or an anti-HER2 antibody are used as the parent antibodies (first or second antibody) to generate the molecule of Format 2 or Format 3. In one aspect, the created molecule is a bivalent and bi-specific molecule recognizing two different epitopes on HER1 (or HER2), or recognizing HER1 at one binding site and HER2 at the other binding site. In another aspect, the created molecule is a bivalent, mono-specific molecule recognizing same epitope of HER1 (or HER2) at both binding sites.

The HER molecule (e.g. HER antibody-like molecule) is produced with the methods of the present invention and described in Section E. In particular, to produce the HER molecule of Format 1 or Format 2, the gene encoding the heavy chain (or partial heavy chain as in Format 2) of the first HER antibody is connected with the gene encoding the VH or VL domain(s) of the second antibody so that a DNA construct encoding a hybrid heavy chain is so created. The DNA construct is then put into the expression vector described herein, which is co-transfected into a host cell described herein with an expression vector comprising the gene encoding the light chain of the first antibody. The desired molecule is then expressed and assembled in the host cell.

In an exemplary embodiment, where an anti-HER1 antibody is used as the first antibody and anti-HER2 antibody is used as the second antibody for a molecule of Format 1, a DNA construct is produced comprising a nucleic acid molecule encoding a single heavy chain of an anti-Her 1 antibody connected at its 3′ end to a nucleic acid molecule encoding a linker further functionally connected with a nucleic acid molecule encoding a VH or a tandem of VHs of an anti-Her2 antibody. Another DNA construct is produced comprising a nucleic acid molecule encoding a single heavy chain of an anti-HER1 antibody fused at its 3′ end to a nucleic acid molecule encoding a linker that is further functionally connected with a nucleic acid molecule encoding a VL or a tandem of VLs of the anti-HER2 antibody. The produced two DNA constructs are introduced into separate expression vector and co-transfected into a host cell together with an expression vector comprising nucleic acid molecule encoding the light chain of the anti-HER1 antibody. The HER molecule is then assembled and secreted.

In another exemplary embodiment, where an anti-HER2 antibody is used as the first antibody and anti-HER1 antibody is used as the second antibody for a molecule of Format 1, a similar DNA construct is produced comprising a nucleic acid molecule encoding a single heavy chain of an anti-HER2 antibody connected at its 3′ end to nucleic acid molecule encoding a linker further functionally connected with a nucleic acid molecule encoding a VH or VL or a tandem of VHs or VHs of an anti-HER1 antibody. The produced two DNA constructs are introduced into separate expression vector and co-transfected into a host cell together with an expression vector comprising nucleic acid molecule encoding the light chain of the anti-HER 1 antibody. The HER multi-specific molecule is then assembled and secreted.

In order to provide optimal conditions for assembly of the molecules of Format 1 or 2, the expression vector comprising the DNA construct encoding the heavy chain and the vector comprising the DNA construct encoding the light chain are co-transfected at a ratio of 5:1; 4:1; 3:1; 2:1; 1:1:2, 1:3, 1:4; or 1:5, preferably 1:1. In an preferred embodiment, the co-transfection ratio is 1:1:2 for the expressing vector comprising the DNA construct of HER1 (or HER2) antibody heavy chain connected with VH or a tandem of VH: the expressing vector comprising the DNA construct encoding HER1(or HER2) antibody heavy chain connected with VL or a tandem of VL: the expressing vector comprising the DNA construct encoding the light chain of the anti-HER1 (or HER2) antibody.

To produce the molecule of Format 3, a DNA construct comprising a nucleic acid molecule encoding a single chain of the molecule of Format 3 is generated. Such a single chain comprises a fragment of CH2-CH3 having the CH2 domain connected at its N-terminus to a VH (or VL) of an anti-HER1 (or HER2) antibody and having the CH3 domain connected at its C-terminus to a VH (or VL) of an anti-HER2 (or HER1) antibody. The DNA construct is introduced into an expression vector, which is further transfected into a host cell. The desired molecule is then expressed and assembled in the host cell.

The variable and/or constant regions of the hybrid heavy chain of the HER multi-specific molecule can also be modified with the approaches described herein, for example, to enhance hetero-dimerization.

In one specific embodiment, the HER1 antibody comprises six CDRs (CDR1, CDR2, CDR3, CDR4, CDR5 and CDR6), having an amino acid sequence of SEQ ID NO: 1, 2, 3, 4, 5, and 6 respectively.

In a more specific embodiment, the HER1 antibody comprises a heavy chain variable region comprising an amino acid sequence of SEQ ID NO:7 and a light chain variable region comprising an amino acid sequence of SEQ ID NO:8.

In another specific embodiment, the HER2 antibody comprises six CDRs (CDR1, CDR2, CDR3, CDR4, CDR5 and CDR6), having an amino acid sequence of SEQ ID NO: 9, 10, 11, 12, 13, and 14 respectively.

In yet another specific embodiment, the HER2 antibody comprises a heavy chain variable region comprising an amino acid sequence of SEQ ID NO:15 and a light chain variable region comprising an amino acid sequence of SEQ ID NO:16.

Variant antibodies of the above described HER1 and HER2 antibodies can also be used as antibodies for producing HER multi-specific molecules. These variants include antibodies that have been mutated, yet have at least 60%, 70%, 80%, 90%, 95%, 98% or 99% identity in the CDR regions with the CDRs of the above HER1 or HER2 antibodies. In some embodiments, it includes mutant antibodies wherein no more than 1, 2, 3, 4 or 5 amino acids have been mutated in the CDR regions when compared with SEQ ID NO: 1, 2, 3, 4, 5, 6, or 9, 10, 11, 12, 13, or 14, while still maintaining their specificity for the original antibody's epitope.

Other variant antibodies of the invention include antibodies that have been mutated in the framework regions, yet have a heavy variable region having a sequence at least 60%, 70%, 80%, 90%, 95%, 98%, 99% identical with SEQ ID NO: 7 (for HER1 antibody variants) or 15 (for HER2 variants), and a light chain variable region having a sequence at least 60%, 70%, 80%, 90%, 95%, 98% or 99% identical with SEQ ID NO: 8 (for HER1 variants) or 16 (for HER2 variants), while still maintaining their specificity for the original antibody's epitope.

Variant antibodies can be produced by amino acid deletion, insertion or substitution via mutagenesis (e.g., site-directed or PCR-mediated mutagenesis) of nucleic acid molecules encoding SEQ ID NOs: 1-14 respectively, followed by testing of the encoded altered antibody for retained function using the functional assays described herein.

In one exemplary embodiment, the present invention includes a molecule comprising two hybrid heavy chains. At least one hybrid heavy chain comprises a heavy chain variable region having an amino acid sequence at least 60%, 70%, 80%, 90%, 95%, 98%, 99% or 100% identical to SEQ ID NO:7 (or SEQ ID NO:15) and connected at the C-terminus of its constant region with a VH having a an amino acid sequence at least 60, 70, 80, 90, 95, 98%, 99% or 100% identical to SEQ ID NO:15 (or SEQ ID NO:7) or a tandem thereof. Preferably, the other hybrid heavy chain comprises a N-terminal heavy chain variable region having an amino acid sequence at least 60%, 70%, 80%, 90%, 95%, 98%, 99% or 100% identical to SEQ ID NO:7 (or SEQ ID NO:15) and connected at the C-terminus of its constant region with a VL having a an amino acid sequence at least 60%, 70%, 80%, 90%, 95%, 98%, 99%, or 100% identical to SEQ ID NO:16 (or SEQ ID NO:8) or a tandem thereof.

The present application also includes a HER1/HER2 multi-specific molecules that have been modified at the CH3 domains in both heavy chains (A chain and B chain) using the methods described in Section H of the present application. The present invention includes a polypeptide having an amino acid sequence at least 60%, 70%, 80%, 90%, 95%, 98%, 99% or 100% identical to SEQ ID NO:17 (or SEQ ID NO:18), as well as a nucleic acid molecules having nucleotide sequences 60%, 70%, 80%, 90%, 95%, 96%, 97%, 98%, 99% or 100% identical to the sequences encoding SEQ ID NO: 19 or 20.

The present application also includes expressing vectors comprising the above-described nucleic acid sequences.

The present application further includes host cells comprising the above expression vectors.

The produced HER1/HER2 (or HER2/HER1) multi-specific molecules are useful in both diagnosis and treatment of cancer. Preferred cancer indications include ovarian cancer; peritoneal cancer; fallopian tube cancer; breast cancer, including metastatic breast cancer (MBC); lung cancer, including non-small cell lung cancer (NSCLC); prostate cancer; colorectal cancer; and/or cancer which displays HER expression, amplification and/or activation.

Aside from cancer, the HER multi-specific molecules as disclosed herein may be used to treat various non-malignant diseases or disorders. Such non-malignant diseases or disorders include autoimmune disease, endometriosis; scleroderma; restenosis; polyps such as colon polyps, nasal polyps or gastrointestinal polyps; fibroadenoma; respiratory disease, etc.

Where the disease is cancer, the patient may be treated with a combination of the HER antibody, and one or more chemotherapeutic agent(s). Preferably at least one of the chemotherapeutic agents is an antimetabolite chemotherapeutic agent such as gemcitabine. The combined administration includes coadministration or concurrent administration, using separate formulations or a single pharmaceutical formulation, and consecutive administration in either order, wherein preferably there is a time period while both (or all) active agents simultaneously exert their biological activities. Thus, the antimetabolite chemotherapeutic agent may be administered prior to, or following, administration of the HER multi-specific molecule. In this embodiment, the timing between at least one administration of the antimetabolite chemotherapeutic agent and at least one administration of the HER multi-specific molecule is preferably approximately 1 month or less, and most preferably approximately 2 weeks or less. Alternatively, the antimetabolite chemotherapeutic agent and the HER multi-specific molecule are administered concurrently to the patient, in a single formulation or separate formulations. Treatment with the combination of the chemotherapeutic agent (e.g. antimetabolite chemotherapeutic agent such as gemcitabine) and the HER multi-specific molecule may result in a synergistic, or greater than additive, therapeutic benefit to the patient.

The HER multi-specific molecule is administered to a human patient in accord with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. Intravenous administration of the antibody is preferred.

For the prevention or treatment of disease, the dose of HER multi-specific molecule will depend on the type of disease to be treated, as defined above, the severity and course of the disease, whether the molecule is administered for preventive or therapeutic purposes, previous therapy, the patient's clinical history and response to the antibody, and the discretion of the attending physician. The fixed dose is suitably administered to the patient at one time or over a series of treatments. Preferably, the dose is in the range from about 20 mg to about 2000 mg of the HER multi-specific molecule.

Where a series of doses are administered, these may, for example, be administered approximately every week, approximately every 2 weeks, approximately every 3 weeks, or approximately every 4 weeks, but preferably approximately every 3 weeks. The doses may, for example, continue to be administered until disease progression, adverse event, or other time as determined by the physician. For example, from about two, three, or four, up to about 17 or more fixed doses may be administered.

Aside from the HER multi-specific molecule and antimetabolite chemotherapeutic agent, other therapeutic regimens may be combined therewith. For example, a second (third, fourth, etc) chemotherapeutic agent(s) may be administered, wherein the second chemotherapeutic agent is either another, different antimetabolite chemotherapeutic agent, or a chemotherapeutic agent that is not an antimetabolite. For example, the second chemotherapeutic agent may be a taxane (such as paclitaxel or docetaxel), capecitabine, or platinum-based chemotherapeutic agent (such as carboplatin, cisplatin, or oxaliplatin), anthracycline (such as doxorubicin, including, liposomal doxorubicin), topotecan, pemetrexed, vinca alkaloid (such as vinorelbine), and TLK 286. “Cocktails” of different chemotherapeutic agents may be administered.

In addition to the above therapeutic regimes, the patient may be subjected to surgical removal of cancer cells and/or radiation therapy.

The foregoing written specification is considered to be sufficient to enable one skilled in the art to practice the invention. The present invention is not to be limited in scope by the constructs deposited, since the deposited embodiments are intended to illustrate only certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims. Indeed, various modifications of the invention in addition to those shown and described herein will become apparent to those skilled in the art from the foregoing description and fall within the scope of the appended claims.

It is understood that the application of the teachings of the present invention to a specific problem or situation will be within the capabilities of one having ordinary skill in the art in light of the teachings contained herein.

Further details of the invention are illustrated by the following non-limiting Examples. The disclosures of all citations in the specification are expressly incorporated herein by reference.

### EXAMPLES

The examples describe the design, production, and testing of the multi-specific and/or multi-valent molecules of the present invention.

### Example 1

### Design of Bispecific Molecules

The sequences of anti-epidermal growth factor receptor (EGFR) antibody C225 and anti-HER2 receptor antibody have been used in designing the bispecific molecules. The constructs designed and characterized in this study are schematically shown in FIG. 1. Specifically, for format 1A, VH or VL of anti-HER2 receptor antibody has been fused to the C-terminal of CH3 of both heavy chains of C225 full IgG1 antibody to make bispecific trivalent molecule. Heavy chain fused with VH of anti-HER2 receptor antibody is defined as chain A, whereas heavy chain fused with VL of anti-HER2 receptor antibody is defined as chain B. A series of linkers with 0, 1, 2, 3, 4 GGGGS (SEQ ID NO. 21) have been inserted between the C-terminals of CH3 and the N-terminal of VH or VL to test the effect of linker length on binding. The proteins were named 0101-0105 IgG-V. For format 1B, two VH or VL in tandem have been fused to the C-terminal of CH3, with linkers of three different length (0, 1 or 2 GGGGS) in between the two tandem VH or VL. The linker between CH3 and VH or VL was fixed with 3(G4S). The proteins were named 0106-0108 IgG-V.

To promote heterodimerization between chain A and chain B, a total of seven mutations were introduced to CH3 of chain A, three mutations were introduced to CH3 chain B. In order to test the formation and proportion of heterodimerization, we introduced deletion of VH-CH1 in chain B to make one arm molecules. They were used to validate the effect of heterodimerization between CH3 domains. In addition, they are bispecific divalent molecules that could be applied when multivalency is not desirable. All one arm molecules were based on backbone of 0104 IgG-V.

A number of one arm molecules were created. They included control (0104 OA-V), mutant with charge mutations on each CH3 domain (0104 OAA-V), and mutant 0104 OAN-V.

The constructs were made with internally developed vector pRS5a-hIgG1 with CMV promoter.

### CH3 Heterodimerization

The interactions between two chains of CH3 were visually inspected in the X-ray structure of 1H3X to predict mutations for enhancing heterodimerization while reducing homodimerization between two CH3 domains.

Two hydrogen bonds are formed between two pairs of polar residues at the center of the binding interface between the two CH3 domains of the heavy chains (chain A and chain B, respectively): Thr366 of chain A forms a hydrogen bond with Tyr407 of chain B, while Thr366 of chain B forms a hydrogen bond with Tyr407 of chain A. These two hydrogen bonds stabilize the binding interface between chain A and chain B. In order to achieve favorable binding between heterodimer chains while unfavorable binding between homodimer chains at the same time, we postulate to mutate Thr366 of chain A and Tyr407 of chain B to valine and phenylalanine, respectively. In such a configuration, the heterodimer formed by chain A and chain B will have two pairs of favorable interactions in the center. One pair is formed by the hydrophobic interaction between Va1366 of chain A and Phe407 of chain B, the other is formed between the polar interaction between Thr366 of chain A and Tyr407 of chain B. The likelihood to form chain A homodimer is reduced by the unfavorable interaction between the hydrophobic residue Va1366 of one chain A and the polar residue Tyr407 of another chain A. The interaction strength between a pair of chain B is likely to be reduced as well, due to the unmatched interaction between the polar residue Thr366 of one chain B and the hydrophobic residue Phe407 of another chain B.

The polar residue Ser364 at the binding interface does not form any inter-domain hydrogen bonds with residues on the other chain. The residue it faces on the other chain is the hydrophobic residue Leu368. To improve the binding between heterodimer chains without perturbing the interaction between homodimer chains, we propose to mutate Ser364 and Leu368 of chain A to be leucine and glutamine, respectively. When chain A and chain B forms a heterodimer, two pairs of energetically favorable interactions are formed. One pair is between two hydrophobic residues of Leu364 of chain A and Leu368 of chain B. The other pair is between two polar residues of Gln368 of chain A and Ser364 of chain B. In either the chain A and chain A homodimer or the chain B and chain B homodimer, the interaction between residue 364 and 368 is of the same type as that in the wild type protein, which is the energetically unfavorable interaction between a polar residue and a hydrophobic residue.

Two cation-π interactions are formed at the binding interface of two CH3 chains. One pair is between Phe405 of chain A and Lys409 of chain B, and the other between Lys409 of chain B and Phe405 of chain A. Since cation-π interaction is typically weaker than π-π interaction, we speculate that heterodimerization could be facilitated by replacing the cation-π interactions with π-π interaction. Specifically, we propose to mutate Phe405 and Lys409 of chain A to serine and phenylalanine, respectively, and to mutate Lys409 of chain B to glutamine. When chain A binds to chain B, Ser405 of chain A will form a hydrogen bond with Gln409 of chain B, while phe409 of chain A and Phe405 of chain B will form a π-π interaction. Both interactions are energetically favorable, making it easier for chain A and chain B to form a heterodimer. The π-π interaction is a particularly strong interaction, tilting the population balance toward the heterodimer state. At the same time, the formation of homodimers is not reduced by the unfavorable interaction between the hydrophobic phenylalanine residue and a polar residue. In the case of chain A forms a homodimer with another chain A, Phe405 does not interact favorably with Gln409. In the case of chain B forms a homodimer with another chain B, Phe409 does not interact favorably with Ser405.

At the margin of the binding interface between two CH3 domains, the charged residue Asp399 of one chain faces directly at a polar residue Thr411 of another chain. We predict that charge complementarity between these two residues could strengthen the heterodimer interaction while weakening the homodimer interaction. In particular, we propose to mutate both Asp399 and Thr411 of chain A to lysine, and to mutate Thr411 of chain B to aspartic acid. Two pairs of complementary charges thus form salt bridges in the heterodimer form. One pair is between Lys399 of chain A and Asp411 of chain B, while the other pair is between Lys411 of chain A and Asp399 of chain B. Meanwhile, repulsive electrostatic interaction forbids the formation of homodimers. In chain A and chain A homodimer, Lys399 repels Lys411. In chain B and chain B homodimer, Asp399 repels Asp411.

In summary, we mutate seven residues on chain A and three residues on chain B to facilitate heterodimerization between CH3 domains. The mutations on chain A are: Ser364Leu, Thr366Val, Leu368Gln, Asp399Lys, Phe405Ser, Lys409Phe and Thr411Lys. The mutations on chain B are: Tyr407Phe, Lys409Gln and Thr411Asp. Most of these mutations serve one purpose, which is to make residues at the binding interface to interact with like residues in the heterodimer configuration, while with unlike residues in the homodimer configuration. In specific, these mutations are designed so that polar residues interact with polar residues, while hydrophobic residues interact with hydrophobic residues, in the heterodimer formation. In the homodimer formation, residues are mutated so that polar residues interact with hydrophobic residues. The favorable interactions in the heterodimer configuration and the unfavorable interactions in the homodimer configuration work together to make it more likely for CH3 domains to form heterodimers than to form homodimers. This mutation design rational is named as “polar-bridging.”

### Expression, Affinity Purification, Protein Quantatition and Western Blot

All constructs were transiently expressed in HEK293T adherent cells using Lipofectamine-2000 (Invitrogen). HEK 293T cells were co-transfected with C225 light chain and two heavy chains at ratio of 2:1:1. After transfection, the cells were grown in serum free Hyclone media (Thermo Scientific) and the supernatant was collected after 3 days of incubation at 37° C. The supernatants and protein A purified samples were used for Western blot and ELISA analysis. Anti-Fc antibody (Sigma), anti-Fab, anti-Kappa antibody were used for western blot. For small scale purification, 10 ml supernatant was firstly concentrated by Pierce concentrator (20K) and purified by Protein A Nab spin column according to the protocol from manufacture.

### Single or Dual Antigen Binding ELISA

For single antigen binding assay, ELISA maxisorp plate was coated with HER2-Fc at 3 μg/ml in PBS at 4° C. overnight. The plate was wash in washing buffer (PBS/0.05% Tween-20) and blocked with 1% BSA/PBS. Supernatants with bispecific molecules were serially diluted with the blocking buffer at 1 to 3 ratio and were applied to the antigen-coated plate. After incubation for 1 hr, the plate was washed 5 times. Bound molecules were detected by HRP-conjugated anti-Fab antibody (Sigma) and visualized with 3,3′,5,5′-tetramethylbenzidine substrate (R&D). The absorbance at 450 nm was measured using a microplate reader. The resulting data were analyzed using GraphPad Prism 5(GraphPad).

For cross-link ELISA or dual antigen binding assay, the plate was coated with the first antigen either HER2-Fc or EGFR-Fc at 3 μg/ml in PBS at 4° C. overnight. After blocking, purified bispecific molecules in serial dilutions were added for incubation, followed by the addition of the second biotinylated antigen at fixed concentration of 3 μg/ml. Detection was carried out using HRP-conjugated streptavidin (R&D).

### Thermo-Stability Measurement by Differential Scanning Fluorimetry (DSF)

Purified bispecific protein sample of 2.5 μg were mixed with sypro orange (Invitrogen) at 1:1000 in PBS in total volume of 25 μl per well. Using Biorad CFX-96 Q-PCR machine. The samples were firstly incubated at 25° C. for 3 min and then ramp up in temperature at 1 C interval for 0.5 min until reaching 95° C.

The Expression Levels of IgG-V Series with Different Linkers are Comparable to that of One of the Parental Antibody (Her-2 Antibody)

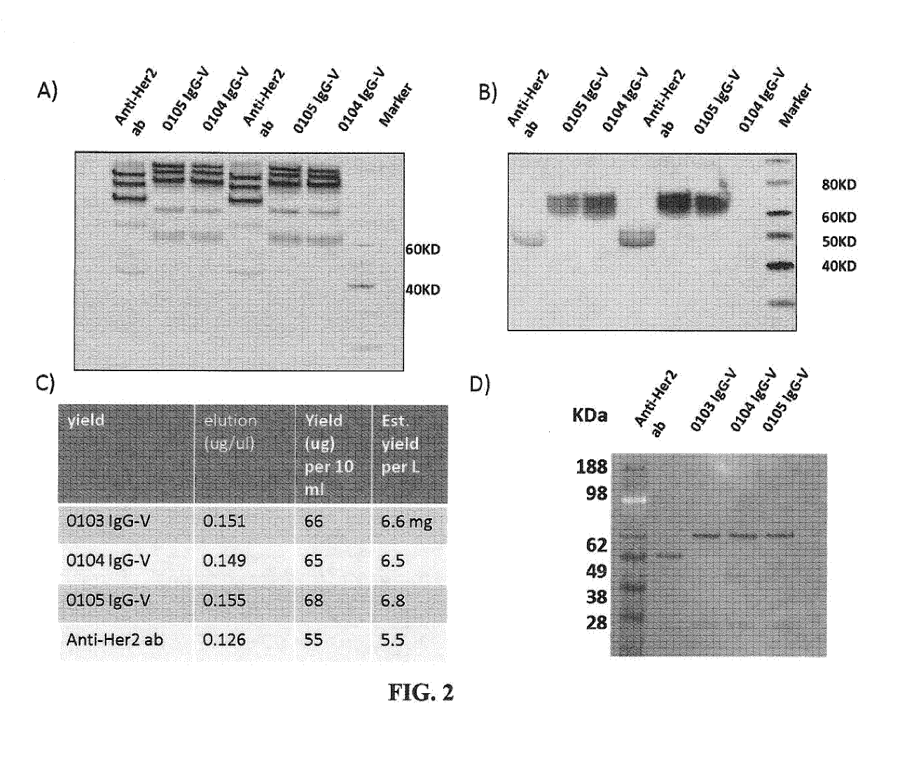

Base on the linker length, the constructs for series 1a were labeled as 0101-0105 IgG-V based on the number of G4S as the linker between CH3 and VH or VL. In Western blot under denaturing condition probed with anti-Fc antibody (FIG. 2B), heavy chains of 0104 IgG-V and 0105 IgG-V showed around size of 62 KD which are consistent with the size prediction. The expression pattern of 0104 or 0105 was similar compared to that of anti-Her2 anitbody under non-denaturing condition, except that they were larger in size (FIG. 2A).

The supernatant was quantified by protein A sensor chip using Qctet Red (Forte Bio). As compared to anti-Her2 antibody, the bi-specific molecules showed comparable or slight increase in yield at around 10 μg/ml of supernatant (FIG. 2C). The 10 ml supernatants of all samples were concentrated by the concentrator with cut off of 20K and then subject to protein A spin column purification. The yield is around 50-60 μg per 10 ml of supernatant. The purity was evaluated by SDS-PAGE under reducing condition (FIG. 2D).

Her2 Binding Site was Reconstituted by VH and VL that were Fused to c-Terminal of Heavy chains and concurrent binding to both antigens

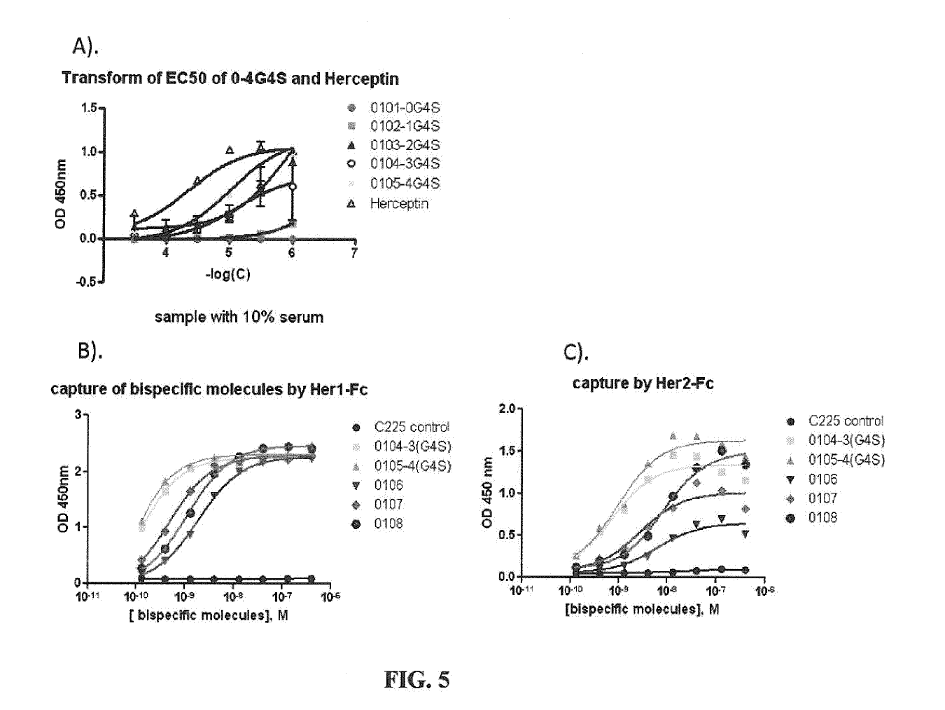

ELISA plate well coated with 3 μg/ml of HER2 Fc was used to capture Her2 binding activity from bi-specific molecules secreted in the cell culture media. The captured bispecific molecules were incubated with anti-Fab-HRP to demonstrate Her2 binding activity. As shown in FIG. 5A, anti-HER2 receptor antibody and 0103, 0104, 0105 showed HER2 receptor binding, while 0101, 0102 showed little HER2 binding activity. It is likely that shorter linkers between CH3 and VH or VL interfere with the orientation and confirmation of VH and VL, therefore affect the reconsitution of the correct binding epitope.

In order to detect dual antigen binding activity of the bispecific molecules, we carried out cross-link ELISA. The first antigen of either EGFR-Fc or HER2-Fc was immobilized onto the ELISA plate. The purified bispecific molecules of serial dilutions were incubated with the first antigen. The unbound material was removed and the second biotinylated antigen of either HER2 or EGFR at fix concentration was added. The concurrent binding to both antigens were shown in FIGS. 5B and 5C. Monospecific antibody C225 didn't show concurrent binding to both antigens.

Of the various linker lengths for 0106, 0107 and 0108, 0106 with no linker between two tandems VH or VL showed the least binding activity. It is interesting to note again that linker length affected HER2 binding activity. However, it remained to be checked whether it is the binding activity from the first pair of VH/VL, the second pair or both that got affected by the shorter linker.

Overall, 0104 and 0105 IgG-V showed the highest binding activity in crosslink ELISA. 0104 IgG-V backbone was chosen for creating one arm molecules.

### Enhancement of Heterodimer Formation by CH3 Mutations

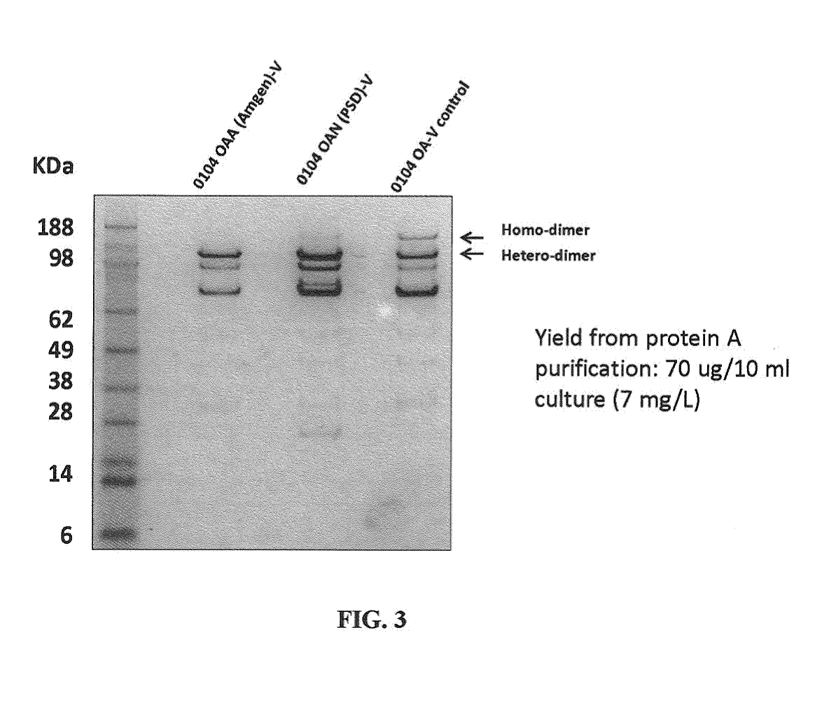

One arm molecules were created and tested for the formation of heterodimerization between CH3 domains of chain A and chain B. The size of heterodimer would be around 125 KDa whereas that of homodimer of chain A would be 175 KDa. As shown in FIG. 3. WT CH3 in 0104 OA there appeared homo-dimer of chain A. CH3 mutations either by charged ones in 0104 OAA or by our combined design in 0104 OAN the band disappeared. This indicated that the mutations in CH3 chain A and chain B promoted the heterodimerization of CH3 chains. Charged mutations in 0104 OAA was used as a control and it also showed the disappearance of the band. From the western blot probed with anti-Kappa chain antibody, it is apparent that homodimer population had been diminished greatly if not completely by mutations in CH3.

### EC50 Determination of Bi-Specific Molecules

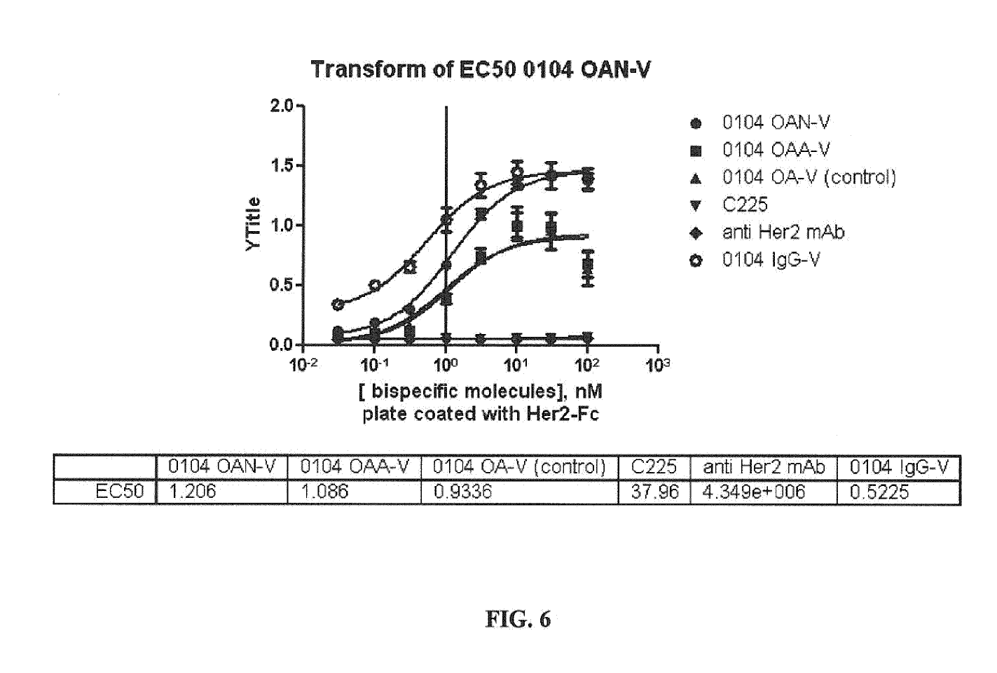

Bispecific binding of 0104 one arm molecules were confirmed in the crosslink ELISA, as shown in FIG. 6. EC50 against Her2-Fc were determined for the one arm molecules, as shown in the table.

Bi-Specific Molecules Showed Similar Thermo-Stability as Compared to that of Parental Antibody C225

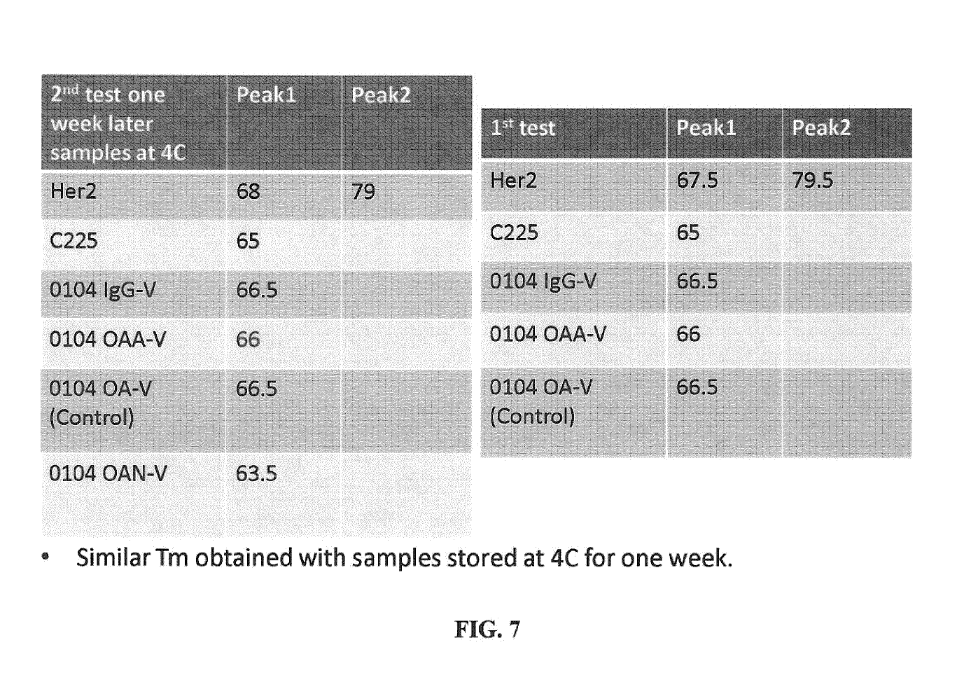

The proteins during temperature increase will experience denaturing and expose hydrophobic residues. The binding of Sypro orange to the hydrophobic residues of denatured proteins will increase fluorescence at 610 nm. The mid-point of the protein unfolding is defined as Tm. As seen from C225, the peak of 68° C. likely represents CH2 and Fab, and the peak of 78° C. which was barely visible could be from CH3 (FIGS. 7A and B). The bispecific molecules showed similar thermo-stability as measured by Tm. There is no difference in Tm with molecules of varying linker length.

### CONCLUSION

The new formats of bi-specific molecules of the present invention demonstrate bi-specific functionality. They were able to bind both targets, EGFR and HER2. VH and VL fused to C-terminal of CH3 were able to re-associate to form the binding moiety for the target, in this case Her2. The shorter length of linker may affect the interactions between VH and VL in a way to affect the binding area reconstitution, as reflected in 0, 1 or 2 G4S. With longer linker 3 or 4 G4S there appeared to be sufficient for binding of Her2. There was no significant difference between 3 or 4 G4S.

In order to facilitate heterodimerization between CH3 in Fc to enrich the population of VH/VL pairs but not VH/VH or VL/VL pairs, we engineered CH3 of chain A and B so interaction of chain A and B is promoted whereas chain A homodimer and chain B homodimer interactions are minimized. As shown by regular SDS-PAGE and western blot that majority of homodimer of chain A was diminished with both charged mutations from Amgen's design and the mutations from our design. When probed with anti-Kappa antibody, one major band was chain A and chain B heterodimers. This indicated that it is possible to purify chain A and B heterodimers using anti-Kappa antibody.

Dual binding for Her1 and Her2 has been demonstrated by crosslink ELISA for 0104 one arm series as well as 0104 IgG-V. EC50 showed that 0104 OAN-V is around 1 nM for Her2 binding.

Expression of IgG-V series and 0104 one arm series looked comparable to that of C225 and anti-Her2 mAb according to regular SDS-PAGE or western blot. The protein A purified yield of 0104 OAN-V is estimated to be around 7 mg/L.

Thermostability measured by thermofluor showed that Tm of 0104 IgG-V is around 66.5 C. This is slightly higher than that of C225 which is 65° C. It is likely that the association of VH and VL at the C-terminal of Fc would further improve thermostability of the whole molecule, as the packing of Fc could be enhanced. Tm of 0104 OAN is around 63° C. The drop in Tm could be due to engineering of CH3 in chain A and B. However this drop is not significant and therefore may not affect the overall property of the molecule.

### Example 2

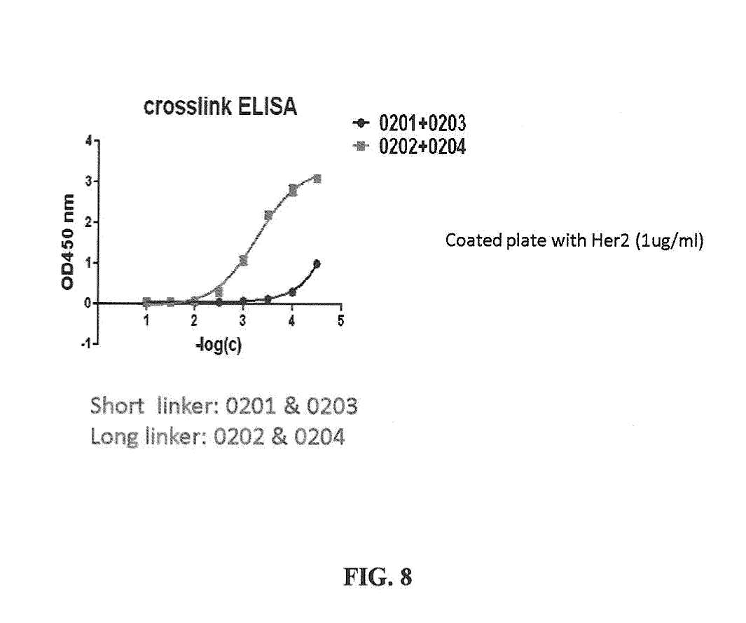

Format 4 (FIG. 1E) was created by fusing VH or VL to the c-terminals of the light chain or heavy chain of a Fab molecule with the first 10 amino acids of the hinge region (upper hinge with one Cysteine retained). The first binding moiety was provided by Her1 antibody; whereas the second binding moiety was provided by the re-associated VH and VL of the Her2 antibody. G₄S linkers with various length were tested. The 3 G₄S and 4 G₄S pair but not the 1 G₄S and 2 G₄S pair resulted in binding activity for the Her2 binding, as shown in FIG. 8.

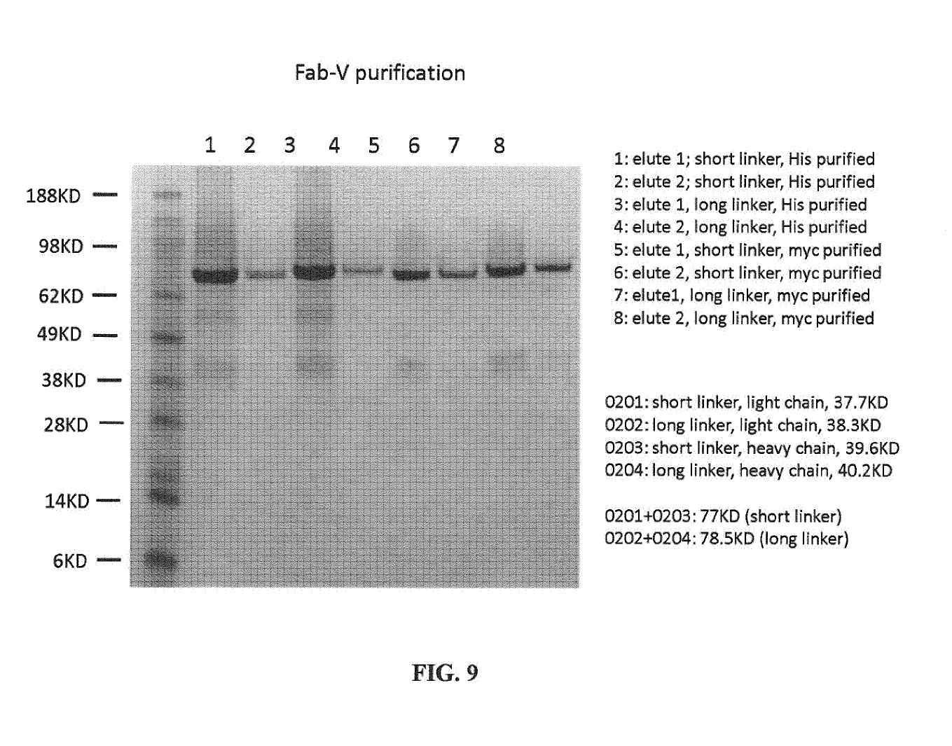

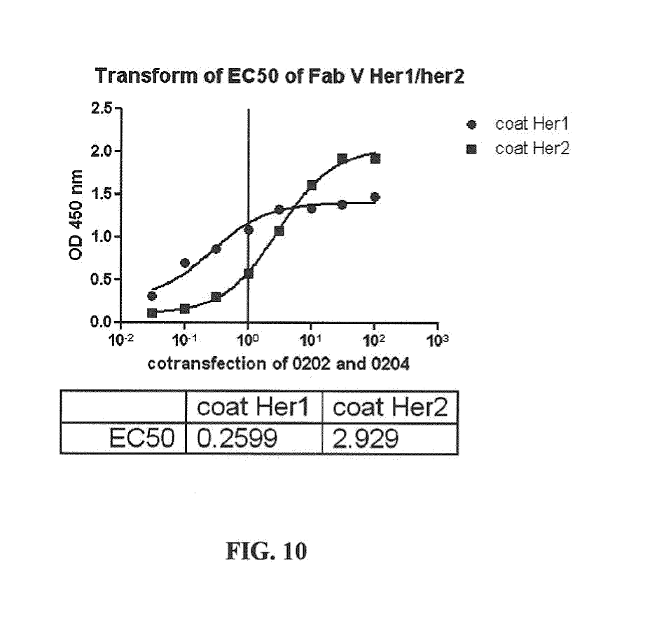

This bispecific molecule can be purified through either His tag which was fused to VH of Her2 antibody or c-myc tag which was fused to VL of Her2 antibody, as shown in FIG. 9. The purified molecule showed one band, which suggested that disulfide bond formation in the upper hinge region is complete. The molecule showed Tm of 68° C. Crosslink ELISA was used to determine the EC50 for both binding activities. As shown in FIG. 10, for Her1 binding, EC50 is 0.26 nM, for Her2 is 2.9 nM, which is comparable to that of scFv of Her2.

This example demonstrated that C-terminal fusions of VH and VL can not only reconstitute binding activities but also may yield a more stable bispecific bivalent molecule.

1. A molecule comprising a Fab region of a first antibody, wherein the CH1 domain of said Fab region connects at its C-terminus with at least the first 5 amino acids of the hinge region of said first antibody, which in turn connects to a VH region of a second antibody via a first linker, and the CL domain of said Fab region connects at its C-terminus a VL region of said second antibody via a second linker.

2. The molecule of claim 1, wherein said Fab region of said first antibody forms a first antigen binding site, and said VH and VL regions of said second antibody forms a second antigen binding site, and wherein said first antigen binding site and second antigen binding site bind to the same antigen.

3. The molecule of claim 2, wherein said first antigen binding site and second antigen binding site bind to the same epitope of said antigen.

4. The molecule of claim 2, wherein said first antigen binding site and second antigen binding site bind to different epitopes of said antigen.

5. The molecule of claim 1, wherein said Fab region of said first antibody forms a first antigen binding site, and said VH and VL regions of said second antibody forms a second antigen binding site, and wherein said first antigen binding site and second antigen binding site bind to two different antigens.

6. The molecule of claim 5, wherein said molecule binds specifically to HER1 and HER2.

7. The molecule of claim 1, wherein said first linker and second linker are a (G₄S)n linker, wherein n is at least 3.

8. A molecule comprising a first antibody comprising one light chain and two heavy chains, wherein one heavy chain includes a variable region and a constant region having a CH1 domain, hinge domain, a CH2 domain, and a CH3 domain, while the other heavy chain consists essentially of a hinge domain, a CH2 domain, and a CH3 domain; wherein the CH3 domain of one of said two heavy chains is connected at its C-terminus to a heavy chain variable region (VH) of a second antibody, and that CH3 domain of the other heavy chain is connected at its C-terminus to a light chain variable region (VL) of said second antibody.

9. The molecule of claim 8, wherein said molecule is modified by introducing into one of the CH3 domains one or more mutations selected from a group consisting of: Ser364Leu, Thr366Val, Leu368Gln, Asp399Lys, Phe405Ser, Lys409Phe and Thr411Lys.

10. A molecule comprising a first antibody having two light chains and two heavy chains with each of the two heavy chains comprising a variable region and a constant region having a CH1 domain, hinge domain, a CH2 domain, and a CH3 domain comprising a N-terminus and a C-terminus; wherein the CH3 domain of one of said heavy chains is connected at its C-terminus to a heavy chain variable region (VH) of a second antibody, and the CH3 domain of the other heavy chain is connected at its C-terminus to a light chain variable region (VL) of said second antibody; wherein said VH and VL of said second antibody are separate peptides; and wherein said molecule is modified by introducing into one of the CH3 domains one or more mutations selected from a group consisting of: Ser364Leu, Thr366Val, Leu368Gln, Asp399Lys, Phe405Ser, Lys409Phe and Thr411Lys.

11. The molecule of claim 10, wherein said VH or VL comprises a N-terminus and a C-terminus, and wherein the C-terminus of said VH is further connected with the N-terminus of a VH of said second antibody while the C-terminus of the VL is further connected with the N-terminus of a VL of said second antibody.

12. A molecule comprising a pair of antibody fragments, each of which consisting of a CH2 domain connected at its C-terminus with a CH3 domain (CH2-CH3); wherein one CH2 domain is connected at its N-terminus to a heavy chain variable region (VH) of a first antibody, and the other CH2 domain is connected at its N-terminus to a light chain variable region (VL) of said first antibody; and wherein one CH3 domain is connected at its C-terminus to a heavy chain variable region (VH) of a second antibody, and the other CH3 domain is connected at its C-terminus to a light chain variable region (VL) of said second antibody; and wherein said molecule is modified by introducing into one of the CH3 domains one or more mutations selected from a group consisting of: Ser364Leu, Thr366Val, Leu368Gln, Asp399Lys, Phe405Ser, Lys409Phe and Thr411Lys.

13. The molecule of claim 9 wherein said molecule is further modified by introducing into the other CH3 domain one or more mutations selected from a group consisting of: Tyr407Phe, Lys409Gln and Thr411Asp.

14. The molecule of claim 8 wherein said molecule binds to both HER1 and HER2.

15. A method of enhancing hetero-dimerization of two heterologous polypeptides, each of which comprises a CH3 domain of an IgG, comprising introducing into one said CH3 domain one or more mutations selected from a group consisting of: Ser364Leu, Thr366Val, Leu368Gln, Asp399Lys, Phe405Ser, Lys409Phe and Thr411Lys, and/or introducing into the other said CH3 domain one or more mutations selected from a group consisting of: Tyr407Phe, Lys409Gln and Thr411Asp.


SEQUENCE LISTING (21 sequences)

SEQ ID NO: 1 - protein, 5 aa, Artificial Sequence
  Humanized
YGVHW

SEQ ID NO: 2 - protein, 21 aa, Artificial Sequence
  Humanized
VIWSGGNTDYNTPFTSRLSIN

SEQ ID NO: 3 - protein, 11 aa, Artificial Sequence
  Humanized
ALTYYDYEFAY

SEQ ID NO: 4 - protein, 11 aa, Artificial Sequence
  Humanized
RASQSIGTNIH

SEQ ID NO: 5 - protein, 7 aa, Artificial Sequence
  Humanized
YASESIS

SEQ ID NO: 6 - protein, 9 aa, Artificial Sequence
  Humanized
QQNNNWPTT

SEQ ID NO: 7 - protein, 119 aa, Artificial Sequence
  Humanized
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 8 - protein, 107 aa, Artificial Sequence
  Humanized
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 9 - protein, 5 aa, Artificial Sequence
  Humanized
DTYIH

SEQ ID NO: 10 - protein, 26 aa, Artificial Sequence
  Humanized
RIYPTNGYTRYADSVKGRFTISADTS

SEQ ID NO: 11 - protein, 11 aa, Artificial Sequence
  Humanized
WGGDGFYAMDY

SEQ ID NO: 12 - protein, 11 aa, Artificial Sequence
  Humanized
RASQDVNTAVA

SEQ ID NO: 13 - protein, 7 aa, Artificial Sequence
  Humanized
SASFLYS

SEQ ID NO: 14 - protein, 9 aa, Artificial Sequence
  Humanized
QQHYTTPPT

SEQ ID NO: 15 - protein, 120 aa, Artificial Sequence [sequence omitted]

SEQ ID NO: 16 - protein, 107 aa, Artificial Sequence [sequence omitted]

SEQ ID NO: 17 - protein, 588 aa, Artificial Sequence
  Humanized
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEFEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVLLVCQVKGFYPSDIAVEWESNGQPENNYKTTPPVLKSDGSFSLYSFLKVDKSRWQQGNVFSCSVMHEALHNHYTQKSLSLSPGKGGGGSGGGGSGGGGSGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 18 - protein, 558 aa, Artificial Sequence
  Humanized
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLFSQLDVDKSRWQQGNVFSCSVMHEALHNHYTQKSLSLSPGKGGGGSGGGGSGGGGSGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 19 - DNA, 1764 bp, Artificial Sequence
  Humanized
caggtgcagctgaagcagagcggccctggcctggtgcagccctctcagagcctgagcatcacctgtaccgtgagcggcttcagcctgaccaattacggcgtgcattgggtgcggcagtctccaggcaagggcctggaatggctgggagtgatctggtccggcggcaacaccgactacaacacccccttcaccagcagactgagcatcaacaaggacaacagcaagagccaggtgttcttcaagatgaacagcctgcagagcaacgacaccgccatctactattgtgccagggccctgacctactacgactacgagttcgcctactggggccagggcaccctggtgaccgtgagcgccgctagcaccaagggccccagcgtgttccccctggcccccagcagcaagagcaccagcggcggcacagccgccctgggctgcctggtgaaggactacttccccgagcctgtgaccgtgtcctggaatagcggagccctgacctccggcgtgcacaccttccccgccgtgctgcagagcagcggcctgtactccctgagcagcgtggtgaccgtgcccagcagcagcctgggcacccagacctacatctgcaacgtgaaccacaagcccagcaacaccaaagtggacaagaaagtggaattcgagcccaagagctgcgataagacccacacctgccccccctgccctgcccccgagctgctgggcggacctagcgtgttcctgttcccccccaagcctaaggacaccctgatgatcagcaggacccccgaagtgacctgcgtggtggtggatgtgagccacgaggaccctgaagtgaagttcaactggtacgtggacggcgtggaagtgcacaacgccaagaccaagcccagagaggagcagtacaacagcacctaccgcgtggtgtctgtgctgaccgtgctgcaccaggattggctgaacggcaaggagtacaagtgcaaagtgagcaacaaggccctgcctgcccctatcgagaaaaccatcagcaaggccaagggccagccacgggagccccaggtgtacaccctgcccccctcccgggaggagatgaccaagaaccaggtgctgctggtgtgtcaggtgaagggcttctaccccagcgacatcgccgtggagtgggagagcaacggccagcccgagaacaactacaagaccacccccccagtgctgaagagcgacggcagcttcagcctgtacagcttcctgaaggtggacaagtccaggtggcagcagggcaacgtgttcagctgcagcgtgatgcacgaggccctgcacaaccactacacccagaagagcctgagcctgtcccccggcaagggtggaggcggttcaggcggaggtggctctggcggtggcggctccggatccgaggttcagctggtggagtctggcggtggcctggtgcagcccgggggctctctccgtttgtcctgtgcagcttctggcttcaacattaaagacacctatatccactgggtgcgtcaggctccgggtaagggcctggagtgggttgcaaggatttatcctacgaatggttatactcgttatgccgatagcgtcaagggccgtttcactataagcgcagacacttcgaaaaacacagcctacctccagatgaacagcctgcgtgctgaggacactgccgtctattattgtagcagatggggtggggacggcttctatgctatggactactggggtcaaggtacactagtcaccgtcagcagc

SEQ ID NO: 20 - DNA, 1674 bp, Artificial Sequence
  Humanized
caggtgcagctgaagcagagcggccctggcctggtgcagccctctcagagcctgagcatcacctgtaccgtgagcggcttcagcctgaccaattacggcgtgcattgggtgcggcagtctccaggcaagggcctggaatggctgggagtgatctggtccggcggcaacaccgactacaacacccccttcaccagcagactgagcatcaacaaggacaacagcaagagccaggtgttcttcaagatgaacagcctgcagagcaacgacaccgccatctactattgtgccagggccctgacctactacgactacgagttcgcctactggggccagggcaccctggtgaccgtgagcgccgctagcaccaagggccccagcgtgttccccctggcccccagcagcaagagcaccagcggcggcacagccgccctgggctgcctggtgaaggactacttccccgagcctgtgaccgtgtcctggaatagcggagccctgacctccggcgtgcacaccttccccgccgtgctgcagagcagcggcctgtactccctgagcagcgtggtgaccgtgcccagcagcagcctgggcacccagacctacatctgcaacgtgaaccacaagcccagcaacaccaaagtggacaagaaagtggcccccgagctgctgggcggacctagcgtgttcctgttcccccccaagcctaaggacaccctgatgatcagcaggacccccgaagtgacctgcgtggtggtggatgtgagccacgaggaccctgaagtgaagttcaactggtacgtggacggcgtggaagtgcacaacgccaagaccaagcccagagaggagcagtacaacagcacctaccgcgtggtgtctgtgctgaccgtgctgcaccaggattggctgaacggcaaggagtacaagtgcaaagtgagcaacaaggccctgcctgcccctatcgagaaaaccatcagcaaggccaagggacaaccaagagaaccccaagtctatactctccctccctcacgagaagaaatgacgaagaaccaggtgtcgttgacatgcctcgttaaagggttctatcccagcgacattgcagtggaatgggagtcaaacgggcaacccgagaataactataagaccacacccccagtgctggacagtgacggttcctttttcttgttcagccagctggacgtggacaaatcgagatggcagcagggaaacgtgttctcctgttccgtaatgcacgaagcccttcataatcattacacccagaagtccctgtcgttgtcgccgggtaaaggtggaggcggttcaggcggaggtggctctggcggtggcggctccggatccgatatccagatgacccagtccccgagctccctgtccgcctctgtgggcgatagggtcactatcacctgccgtgccagtcaggatgtgaatactgctgtagcctggtatcaacagaaacccggaaaggccccgaaactgctgatttactcggcatccttcctctactctggagtcccttctcgcttctctggttcccgctctgggacggatttcactctgaccatcagctccctgcagccggaagacttcgcaacttattactgtcagcaacactatactactcctccgacgttcggacagggtaccaaggtggagatcaaa

SEQ ID NO: 21 - protein, 5 aa, Artificial Sequence
  Linker
GGGGS